SEQUENCE LISTING (26 sequences)

SEQ ID NO: 1 - protein, 20 aa, Artificial Sequence
  Synthetic- Viral L-domain motif
AAPTAPPTGAADSIPPPYSP

SEQ ID NO: 2 - protein, 11 aa, Artificial Sequence
  Synthetic- Viral L-domain motif
TAPSSPPPYEE

SEQ ID NO: 3 - protein, 17 aa, Artificial Sequence
  Synthetic- Viral L-domain motif
MRRVILPTAPPEYMEAI

SEQ ID NO: 4 - protein, 16 aa, Artificial Sequence
  Synthetic- Viral L-domain motif
NTYMQYLNPPPYADHS

SEQ ID NO: 5 - protein, 4 aa, Artificial Sequence
  Synthetic- Viral L-domain motif
PPAY

SEQ ID NO: 6 - protein, 4 aa, Artificial Sequence
  Synthetic- Viral L-domain motif
PPTY

SEQ ID NO: 7 - protein, 4 aa, Artificial Sequence
  Synthetic- Viral L-domain motif
PPPY

SEQ ID NO: 8 - protein, 4 aa, Artificial Sequence
  Synthetic- Viral L-domain motif
PTAP

SEQ ID NO: 9 - protein, 14 aa, Artificial Sequence
  Synthetic- Viral L-domain motif
QSIKAFPIVINSDG

SEQ ID NO: 10 - protein, 11 aa, Artificial Sequence
  Synthetic- Viral L-domain motif
RLNAFPIVMGQ

SEQ ID NO: 11 - protein, 19 aa, Artificial Sequence
  Synthetic- Viral L-domain motif
ATASAPPPPYVGSGLYPSL

SEQ ID NO: 12 - protein, 22 aa, Artificial Sequence
  Synthetic- Viral L-domain motif
PEPTAPPFLQSRPEPTAPPEES

SEQ ID NO: 13 - protein, 14 aa, Artificial Sequence
  Synthetic- Viral L-domain motif
DPQIPPPYVEPTAP

SEQ ID NO: 14 - protein, 11 aa, Artificial Sequence
  Synthetic- Viral L-domain motif
QNLYPDLSEIK

SEQ ID NO: 15 - protein, 21 aa, Artificial Sequence
  Synthetic- Viral L-domain motif
LGIAPPPYEEDTSMEYAPSAP

SEQ ID NO: 16 - protein, 16 aa, Artificial Sequence
  Synthetic- Viral L-domain motif
DDLWLPPPEYVPLKEL

SEQ ID NO: 17 - protein, 48 aa, Artificial Sequence
  Synthetic- Viral L-domain motif
ATASAPPPYVGSGGGATASAPPPYVGSGGGATASAPPPYVGSGGGRRR

SEQ ID NO: 18 - protein, 48 aa, Artificial Sequence; identical to SEQ ID NO: 17
  Synthetic- Viral L-domain motif
  MISC_FEATURE (1)..(1): FITC-MODIFIED N-TERMINUS

SEQ ID NO: 19 - protein, 48 aa, Artificial Sequence; identical to SEQ ID NO: 17
  Synthetic- Viral L-domain motif
  MISC_FEATURE (1)..(1): TAMRA-MODIFIED N-TERMINUS

SEQ ID NO: 20 - protein, 36 aa, Artificial Sequence
  Synthetic- Viral L-domain motif
RPGNFLQSRPEPTAPPFLQSRPEPTAPPEESFRRRR

SEQ ID NO: 21 - protein, 36 aa, Artificial Sequence; identical to SEQ ID NO: 20
  Synthetic- Viral L-domain motif
  MISC_FEATURE (1)..(1): FITC-MODIFIED N-TERMINUS

SEQ ID NO: 22 - protein, 36 aa, Artificial Sequence; identical to SEQ ID NO: 20
  Synthetic- Viral L-domain motif
  MISC_FEATURE (1)..(1): TAMRA-MODIFIED N-TERMINUS

SEQ ID NO: 23 - protein, 879 aa, Artificial Sequence
  WWP2 RECOMBINANT PROTEIN
MDYKDDDDKMASASSSRAGVALPFEKSQLTLKVVSAKPKVHNRQPRINSYVEVAVDGLPSETKKTGKRIGSSELLWNEIIILNVTAQSHLDLKVWSCHTLRNELLGTASVNLSNVLKNNGGKMENMQLTLNLQTENKGSVVSGGELTIFLDGPTVDLGNVPNGSALTDGSQLPSRDSSGTAVAPENRHQPPSTNCFGGRSRTHRHSGASARTTPATGEQSPGARSRHRQPVKNSGHSGLANGTVNDEPTTATDPEEPSVVGVTSPPAAPLSVTPNPNTTSLPAPATPAEGEEPSTSGTQQLPAAAQAPDALPAGWEQRELPNGRVYYVDHNTKTTTWERPLPPGWEKRTDPRGRFYYVDHNTRTTTWQRPTAEYVRNYEQWQSQRNQLQGAMQHFSQRFLYQSSSASTDHDPLGPLPPGWEKRQDNGRVYYVNHNTRTTQWEDPRTQGMIQEPALPPGWEMKYTSEGVRYFVDHNTRTTTFKDPRPGFESGTKQGSPGAYDRSFRWKYHQFRFLCHSNALPSHVKISVSRQTLFEDSFQQIMNMKPYDLRRRLYIIMRGEEGLDYGGIAREWFFLLSHEVLNPMYCLFEYAGKNNYCLQINPASSINPDHLTYFRFIGRFIAMALYHGKFIDTGFTLPFYKRMLNKRPTLKDLESIDPEFYNSIVWIKENNLEECGLELYFIQDMEILGKVTTHELKEGGESIRVTEENKEEYIMLLTDWRFTRGVEEQTKAFLDGFNEVAPLEWLRYFDEKELELMLCGMQEIDMSDWQKSTIYRHYTKNSKQIQWFWQVVKEMDNEKRIRLLQFVTGTCRLPVGGFAELIGSNGPQKFCIDKVGKETWLPRSHTCFNRLDLPPYKSYEQLREKLLYAIEETEGFGQE

SEQ ID NO: 24 - protein, 922 aa, Artificial Sequence
  WWP1 PEPTIDE SEQUENCE
MATASPRSDTSNNHSGRLQLQVTVSSAKLKRKKNWFGTAIYTEVVVDGEITKTAKSSSSSNPKWDEQLTVNVTPQTTLEFQVWSHRTLKADALLGKATIDLKQALLIHNRKLERVKEQLKLSLENKNGIAQTGELTVVLDGLVIEQENITNCSSSPTIEIQENGDALHENGEPSARTTARLAVEGTNGIDNHVPTSTLVQNSCCSYVVNGDNTPSSPSQVAARPKNTPAPKPLASEPADDTVNGESSSFAPTDNASVTGTPVVSEENALSPNCTSTTVEDPPVQEILTSSENNECIPSTSAELESEARSILEPDTSNSRSSSAFEAAKSRQPDGCMDPVRQQSGNANTETLPSGWEQRKDPHGRTYYVDHNTRTTTWERPQPLPPGWERRVDDRRRVYYVDHNTRTTTWQRPTMESVRNFEQWQSQRNQLQGAMQQFNQRYLYSASMLAAENDPYGPLPPGWEKRVDSTDRVYFVNHNTKTTQWEDPRTQGLQNEEPLPEGWEIRYTREGVRYFVDHNTRTTTFKDPRNGKSSVTKGGPQIAYERGFRWKLAHFRYLCQSNALPSHVKINVSRQTLFEDSFQQIMALKPYDLRRRLYVIFRGEEGLDYGGLAREWFFLLSHEVLNPMYCLFEYAGKNNYCLQINPASTINPDHLSYFCFIGRFIAMALFHGKFIDTGFSLPFYKRMLSKKLTIKDLESIDTEFYNSLIWIRDNNIEECGLEMYFSVDMEILGKVTSHDLKLGGSNILVTEENKDEYIGLMTEWRFSRGVQEQTKAFLDGFNEVVPLQWLQYFDEKELEVMLCGMQEVDLADWQRNTVYRHYTRNSKQIIWFWQFVKETDNEVRMRLLQFVTGTCRLPLGGFAELMGSNGPQKFCIEKVGKDTWLPRSHTCFNRLDLPPYKSYEQLKEKLLFAIEETEGFGQE

SEQ ID NO: 25 - protein, 390 aa, Artificial Sequence
  TSG101 PROTEIN SEQUENCE
MAVSESQLKKMVSKYKYRDLTVRETVNVITLYKDLKPVLDSYVFNDGSSRELMNLTGTIPVPYRGNTYNIPICLWLLDTYPYNPPICFVKPTSSMTIKTGKHVDANGKIYLPYLHEWKHPQSDLLGLIQVMIVVFGDEPPVFSRPISASYPPYQATGPPNTSYMPGMPGGISPYPSGYPPNPSGYPGCPYPPGGPYPATTSSQYPSQPPVTTVGPSRDGTISEDTIRASLISAVSDKLRWRMKEEMDRAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 26 - protein, 174 aa, Artificial Sequence
  TRUNCATED-LENGTH TSG101 RECOMBINANT PEPTIDE
MGSSHHHHHHSSGLVPRGSHMASENLYFQGAVSESQLKKMVSKYKYRDLTVRETVNVITLYKDLKPVLDSYVFNDGSSRELMNLTGTIPVPYRGNTYNIPICLWLLDTYPYNPPICFVKPTSSMTIKTGKHVDANGKIYLPYLHEWKHPQSDLLGLIQVMIVVFGDEPPVFSRP

(19) United States
(12) Patent Application Publication
(10) Pub. No.: US 2023/0265069 A1
(43) Pub. Date: Aug. 24, 2023

(54) Small Molecule Inhibitors That Block the Budding of Enveloped Viruses

(71) Applicant: Northwestern University, Evanston, IL (US)

(72) Inventors: Jonathan Leis, Chicago, IL (US); Chi-Hao Luan, Evanston, IL (US); James Edmund Audia, Chicago, IL (US)

(21) Appl. No.: 17/997,013

(22) PCT Filed: Apr. 23, 2021
(86) PCT No.: PCT/US2021/070450

§ 371 Date: Oct. 24, 2022

Related U.S. Application Data
(60) Provisional application No. 63/071,462, filed on Aug. 28, 2020.
(60) Provisional application No. 63/014,418, filed on Apr. 23, 2020.

(51) Int. Cl. C07D 401/12; C07D 401/14; C07D 471/04

(52) U.S. Cl. CPC C07D 401/12; C07D 401/14; C07D 471/04

(57) ABSTRACT

Disclosed are compounds, compositions, and methods for treating an infection by an enveloped virus in a patient in need thereof. The disclosed compounds may be derived from prazole compounds and may inhibit or block the release of an enveloped virus from an infected cell. As such, the disclosed methods may include methods of treating a viral infection by administering the disclosed compounds or pharmaceutical compositions comprising the disclosed compounds.

### CROSS-REFERENCE TO RELATED PATENT APPLICATIONS

This application represents the national stage entry of PCT/US2021/070450 filed on Apr. 23, 2021, which claims the benefit of priority under 35 U.S.C. 119(e) to U.S. Provisional Application No. 63/071,462, filed on Aug. 28, 2020, and U.S. Provisional Application No. 63/014,418, filed on Apr. 23, 2020, the contents of which are incorporated herein by reference in their entireties.

### SEQUENCE LISTING

A Sequence Listing accompanies this application and is submitted as an ASCII text file of the sequence listing named “702581_1949_ST25.txt” which is 28,116 bytes in size and was created on May 14, 2021. The sequence listing is electronically submitted via EFS-Web with the application and is incorporated herein by reference in its entirety.

### BACKGROUND

The invention relates to methods and compositions for blocking the release of enveloped viruses from cells. In particular, the invention relates to methods of administering small molecule derivatives of prazole compounds to treat and/or prevent viral infection and/or disease.

There is considerable interest developing antiviral reagents to combat viral infections. The two most prevalent antiviral strategies focus on creating immunity to viral infection by use of vaccines or by interfering with a necessary virus-specific process essential to virus maintenance, replication and propagation in the host.

Vaccines have been successfully developed for many viruses to combat viral infections. So-called live vaccines containing attenuated version(s) of the target virus provide a convenient means of conferring immunity as typically only one inoculation is required. The drawbacks to most live virus vaccines lie in their limited shelf life, the requirement for maintaining appropriate storage conditions to preserve the vaccine reagent, and the possibility of live attenuating virus vaccines reverting to high virulence due to their active replication. These drawbacks can be avoided by using so-called inactivated virus vaccines containing a completely inert virus particle or a sub-viral component like a protein. The drawback to inactive viral vaccines is that multiple inoculations are required to confer full immunity. Furthermore, vaccines have an attendant risk that adverse reactions might arise in certain populations following immunization (for example, autoimmunity responses associated with Guillain-Barre syndrome (GBS)).

Antiviral compounds that specifically target a viral replication process have also proven effective for treating some virus infections. Examples of such reagents include small molecule inhibitors selective for a given viral protein, such as a viral replicase (e.g., the nucleoside analog 3′-azidothymidine for inhibiting the HIV-1 reverse transcriptase) or a viral protease (e.g., Darunavir for inhibiting HIV-1 protease). Owing to their small molecular size and chemical composition, antiviral compounds can be formulated as pharmaceutical compositions having significant shelf-life and can typically retain their potency over a larger temperature range during storage than many vaccines. However, HIV-1 and other virus can mutate to escape the effectiveness of the antiviral drugs when such drugs are targeted against virus-specific proteins. In particular, HIV-specific drugs have side-effects that cause patients to interrupt therapy that can lead to drug-resistant viral strains.

Generally, antiviral compounds are used in combinations for maximum efficacy and durability. Though most aspects of the viral replication process are susceptible to targeting and inhibition, the primary focus of antiviral inhibitor drug development is on early stage processes of viral replication, when the copy number of viral protein or nucleic acid targets is relatively low.

Late stage replication events include those associated with virus particle assembly and release from the host cell. These viral processes are more difficult targets to develop antiviral reagents against. This is due in part to the vastly larger number of virus particles that result from active viral replication.

Enveloped virus particles adopt an outer membrane structure composed of the host cell membrane in its final virus form. Examples of enveloped viruses include retroviruses (e.g., human immunodeficiency virus, type 1), rhabdoviruses (e.g., rabies virus), herpes viruses (e.g., herpes simplex virus, type 1), and coronaviruses (e.g., sudden acute respiratory syndrome coronavirus type 2 (SARS-CoV-2). For enveloped viruses, the final stages of virus replication include envelope maturation, budding and release from an infected cell.

No antiviral therapeutic reagents have been developed that target the processes of enveloped virus budding and release. This is due in large part to the inability to target virus-specific proteins, owing to the large number of viral proteins present during late phase infection. But more importantly, the host cell-virus interactions responsible for enveloped virus particle maturation, budding and release are only poorly understood. Here, we demonstrate that small molecules can be utilized to inhibit the release of enveloped viruses from infected cells by targeting cellular proteins.

### SUMMARY

Disclosed are compounds, compositions, and methods for treating an infection by an enveloped virus in a subject in need thereof. The disclosed compounds may comprise a prazole moiety or core. The disclosed compounds may inhibit or block release of the enveloped virus from an infected cell. The disclosed compounds may be utilized in methods for treating viral infections.

The disclosed compounds may have a formula as follows:

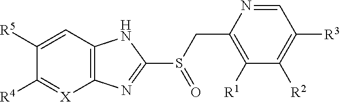

where: X is CH or N; R¹ is H or CH₃; R² is H, methoxy, ethoxy, or methoxypropoxy; R³ is H or CH₃, R⁴ is H, methoxy, N-pyrrolo, pyrazole, triazole, 3-fluoro-pyrrole, imidazole, piperidine, or morpholine; and R⁵ is H, methoxy, or N-pyrrolo. Also contemplated are salts and solvates of the disclosed compounds including pharmaceutically acceptable salts and solvates thereof.

The disclosed compounds may be formulated as pharmaceutical compositions comprising the compounds or pharmaceutically acceptable salts and solvates thereof in a pharmaceutically acceptable carrier for use in treatment methods for a subject in need thereof. In some embodiments, the disclosed compounds and pharmaceutical compositions may be utilized to treat a viral infection by an enveloped virus in a subject. In some embodiments, the disclosed compounds and pharmaceutical compositions may be utilized to inhibit release of an enveloped virus from a cell in an infected subject.

### BRIEF DESCRIPTION OF THE FIGURES

FIG. 1A. Thermal shift data of Tsg101 by compounds Tenatoprazole (N16). The compound caused a dose-dependent shift in the Tₘ for Tsg101-UEV indicating binding to the key domain of Tsg101 as described in Materials and Methods.

FIG. 1B. DHPH, ENO1, and MEK4 do not cause a thermal shift with Tsg101. Thermal shift data of three human proteins not related to Tsg101 by lead compound tenatoprazole. The effect of the prazole compound on the thermal stability of these three proteins is negligible, indicating that the dramatic modulation of the thermal transition of Tsg101 by the prazoles is due to specific interaction.

FIG. 1C. The addition of DTT abolishes the Tₘ shift in the FTS assay, consistent with prazole compounds forming a disulfide bond to Tsg101. Rabe: Rabeprazole, Ila: Ilaprazole, Tenato: tenatoprazole, Panto: pantoprazole, ref: reference.

FIG. 2. Inhibitory effect of tenatoprazole on HSV-2 production and location of virus particles inside of infected cells. Cells with virus were untreated (B1) or treated with 105 tenatoprazole (N16) (B2) for 24 h and examined by transmission electron microscopy. In each case, 80 cells where virus particles were observed were examined. For untreated cells, we observed an average of 120 particles in the cytoplasm ranging from 28 to 204 particles. In the nucleus, we observed an average of 16 particles ranging from 10 to 22 particles. In the presence of drug, we observed a significant increase in dense material in the nucleus with some particles associated with it. We observed an average of 31 particles, ranging from 8 to 48. In the cytoplasm, we did not detect virus particles. Bar=653 1 μm. Inset, higher magnification image.

FIG. 3. Dose-response plots of Tsg101 melting temperature shift caused by 10 prazole compounds. Different concentrations of prazole compounds were incubated with Tsg101 (aa 1-145) and subjected to Fluoresecent Thermal Shift analysis as described in Materials and Methods.

FIG. 4. Inhibitory effect of Ilaprazole on HSV-2 and HSV-1 production. Vero cells infected with HSV-2 or HSV-1 at MOI of 0.1 and examined by transmission electron microscopy 24 h later. HSV-2 infected untreated cells (A & B) and cells treated with 18 μM ilaprazole (C & D). HSV-1 infected untreated cells (E & F) and cells treated with 18 μM ilaprazole (G & H). Eighty cells where virus particles were observed were examined. In the presence of ilaprazole, we observe no or very few particles in the cytoplasm. Treated cells also have an accumulation of electron dense material and particles associated with them. In untreated HSV-2 infected cells, we observed an average of 120 particles ranging from 28-204 in the nucleus, and an average of 16 particles ranging from 10-22 in the nucleus. In treated HSV-2 infected cells, we observed 0 particles in the cytoplasm and an average of 31 particles ranging from 9-56 in the nucleus. In untreated HSV-1 infected cells we observed an average of 135 particles ranging from 12-152 in the cytoplasm, and an average of 35 particles ranging from 10-66 in the nucleus. In treated HSV-1 infected cells, we observed 0 particles in the cytoplasm and an average of 43 particles ranging from 15-166 in the nucleus. Arrows point to virus particles. Nuc, nucleus. Cyt, cytoplasm.

FIG. 5. NMR structure of tenatoprazole metabolite bound to Tsg101. An unoccupied pocket is located adjacent to the pyridine ring and could accommodate substituents for favorable protein contacts. Additional sites for exploration include a small space extending from the pyridine methyl group, and a solvent exposed benzimidazole ring.

FIG. 6A. Fluorescence Thermal Sensitivity graphs of control (DMSO).

FIG. 6B. Fluorescence Thermal Sensitivity graphs of reference compound Ilaprazole.

FIG. 6C. Fluorescence Thermal Sensitivity graphs of reference compound Rabeprazole.

FIG. 6D. Fluorescence Thermal Sensitivity graphs of reference compound Tenatoprazole.

FIG. 7A. Fluorescence Thermal Sensitivity graphs of compound 10.

FIG. 7B. Fluorescence Thermal Sensitivity graphs of compounds 86.

FIG. 7C. Fluorescence Thermal Sensitivity graphs of compounds 48.

FIG. 7D. Fluorescence Thermal Sensitivity graphs of compound 37.

FIG. 7E. Fluorescence Thermal Sensitivity graphs of compound 29.

FIG. 7F. Fluorescence Thermal Sensitivity graphs of compound 47.

FIG. 7G. Fluorescence Thermal Sensitivity graphs of compound 24.

FIG. 7H. Fluorescence Thermal Sensitivity graphs of compound 84.

FIG. 8. Effect of release of HIV-1 from 293T cells by different analogs of Ilaprazole including compound 10, compound 29, and compound 37.

### DETAILED DESCRIPTION

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, “a therapeutic agent” should be interpreted to mean “one or more therapeutic agents” unless the context clearly dictates otherwise. Also, “a substituent” should be interpreted to mean “one or more substituents” unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

New Chemical Entities

New chemical entities may be disclosed herein and may be described using terms known in the art and defined herein.

The term “alkyl” as used herein refers to a saturated straight or branched hydrocarbon, such as a straight or branched group of 1-12, 1-10, or 1-6 carbon atoms, referred to herein as C1-C12 alkyl, C1-C10-alkyl, and C1-C₆-alkyl, respectively.

The term “alkylene” refers to a diradical of an alkyl group. An exemplary alkylene group is —CH₂CH₂—.

The term “haloalkyl” refers to an alkyl group that is substituted with at least one halogen, for example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C2-C12-alkenyl, C2-C10-alkenyl, and C2-C₆-alkenyl, respectively. A “cycloalkene” is a compound having a ring structure (e.g., of 3 or more carbon atoms) and comprising at least one double bond.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C2-C12-alkynyl, C2-C10-alkynyl, and C2-C₆-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C4-8-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of a cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number or ring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C3-C7 heterocyclyl group refers to a saturated or partially unsaturated 3- to 7-membered ring structure containing one to four heteroatoms, such as nitrogen, oxygen, and sulfur. The designation “C3-C7” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical —C(O)—.

The term “carboxy” or “carboxyl” as used herein refers to the radical —COOH or its corresponding salts, e.g. —COONa, etc.

The term “amide” or “amido” or “carboxamido” as used herein refers to a radical of the form —R¹C(O)N(R²)—, —R¹C(O)N(R²) R³—, —C(O)N R² R³, or —C(O)NH₂, wherein R¹, R² and R³ are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Prazole Compounds

Disclosed herein are prazole compounds, pharmaceutical compositions comprising the prazole compounds, and methods of using the prazole compounds and pharmaceutical compositions for treating infections by an enveloped virus in a patient. The disclosed prazole compounds may include compounds or salts or solvates thereof having a formula as follows:

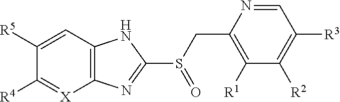

where: X is CH or N; R¹ is H or CH₃; R² is H, methoxy, ethoxy, or methoxypropoxy; R³ is H or CH₃, R⁴ is H, methoxy, N-pyrrolo, pyrazole, triazole, 3-fluoro-pyrrole, imidazole, piperidine, or morpholine; and R⁵ is H, methoxy, or N-pyrrolo. Salts and solvates of the disclosed compounds are also contemplated herein.

In some embodiments, X is CH. In some embodiments, X is N. In some embodiments, R¹ is CH₃. In some embodiments, R² is methoxy. In some embodiments, R³ is CH₃.

In some embodiments, the disclosed compounds may include compounds selected from the group consisting of:

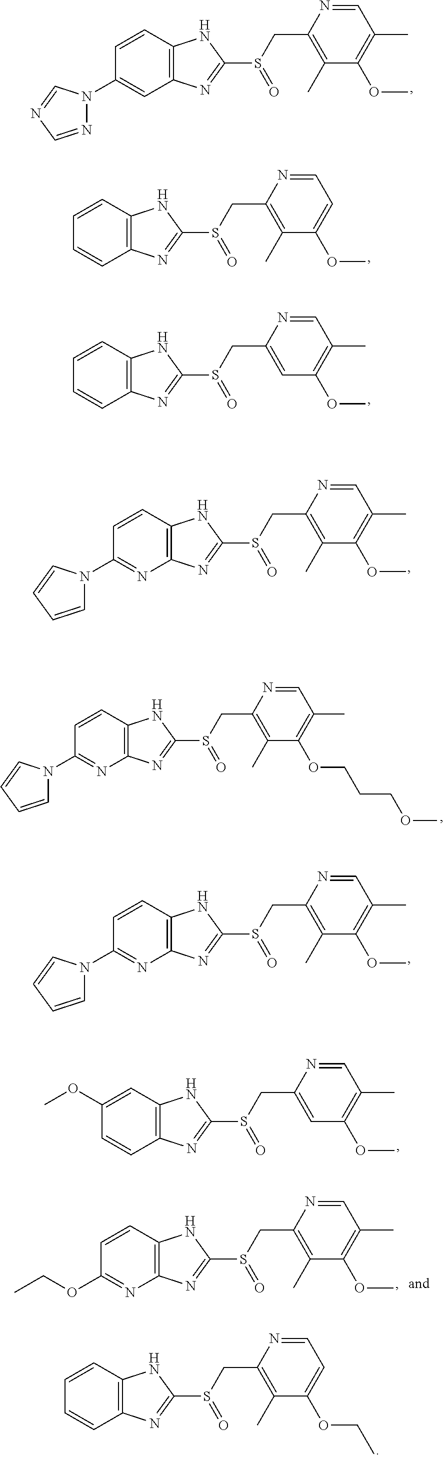

Salts and solvates of the foregoing compounds also are contemplated herein.

Pharmaceutical Compositions

The disclosed prazole compounds may be formulated as therapeutics for treating viral infections and diseases associated with viral infections. The compounds utilized in the methods disclosed herein may be formulated as pharmaceutical compositions that include: (a) a therapeutically effective amount of one or more compounds as disclosed herein; and (b) one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to about 1000 mg/kg body weight (preferably about 0.5 to about 500 mg/kg body weight, more preferably about 50 to about 100 mg/kg body weight). In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 μM, 0.005 μM, 0.01 μM, 0.5 μM, 0.1 μM, 1.0 μM, 10 μM, and 100 μM (e.g., 0.1 μM-1.0 μM).

In some embodiments of the disclosed treatment methods, the subject may be administered a dose of a compound as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of a compound as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds may include doses falling within dose ranges having as end-points any of these disclosed doses (e.g., 2.5 mg-200 mg).

In some embodiments, a minimal dose level of a compound for achieving therapy in the disclosed methods of treatment may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. In some embodiments, a maximal dose level of a compound for achieving therapy in the disclosed methods of treatment may not exceed about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. Minimal and/or maximal dose levels of the compounds for achieving therapy in the disclosed methods of treatment may include dose levels falling within ranges having as end-points any of these disclosed dose levels (e.g., 500-2000 ng/kg body weight of the subject).

The compounds utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The disclosed compounds or pharmaceutical compositions comprising the disclosed compounds may be administered in methods of treatment. For example, the disclosed compounds or pharmaceutical compositions comprising the disclosed compounds may be administered in methods of treating viral infections and/or the symptoms thereof.

Optionally, the disclosed compounds or pharmaceutical compositions comprising the disclosed compounds may be administered with additional therapeutic agents, optionally in combination, in order to treat viral infections. In some embodiments of the disclosed methods, one or more additional therapeutic agents are administered with the disclosed compounds or with pharmaceutical compositions comprising the disclosed compounds, where the additional therapeutic agent is administered prior to, concurrently with, or after administering the disclosed compounds or the pharmaceutical compositions comprising the disclosed compounds. In some embodiments, the disclosed pharmaceutical composition are formulated to comprise the disclosed compounds and further to comprise one or more additional therapeutic agents, for example, one or more additional therapeutic agents for treating viral infections.

Small Molecules for Inhibiting Release of Enveloped Viruses from Infected Cells

Disclosed are methods for treating an infection by an enveloped virus in a patient in need thereof. The methods include administering to the patient a compound that inhibits or blocks release of the enveloped virus from an infected cell. As such, the disclosed methods include methods of inhibiting or blocking release of an enveloped virus from a cell. In some embodiments, the disclosed methods comprise administering to the patient a small molecule inhibitor as disclosed herein.

The disclosed methods may be performed in order treat infection by an enveloped virus in a patient in need thereof, for example, by inhibiting or blocking release of an enveloped virus in infected cells using this common mechanism. Suitable enveloped viruses for the disclosed methods may include, but are not limited to Lassa fever virus, lymphocytic choriomeningitis virus, Ebola virus, Marburg virus, hepatitis B virus, herpes simplex virus type 1, herpes simplex virus type 2, cytomegalovirus, simian virus type 5, mumps virus, avian sarcoma leucosis virus, human immunodeficiency virus type 1, human T-lymphotrophic virus type 1, equine infectious anemia virus, vesicular stomatitis virus, rabies virus, coronavirus (e.g., sudden acute respiratory syndrome virus (SARS-CoV and particular SARS-CoV-2), and combinations thereof.

In some embodiments, the disclosed methods may be performed in order treat infection by coronavirus in a patient in need thereof, for example, by administering a proton pump inhibitor to the patient. Coronaviruses are known in the art and may include human coronavirus, including the human coronaviruses that cause middle east respiratory syndrome (MERS) and sudden acute respiratory syndrome coronavirus (SARS-CoV) including SARS-CoV-2 which causes coronavirus virus infectious disease 2019 (COVID-19). Coronaviruses make up the subfamily Orthocoronavirinae, in the family Coronaviridae, order Nidovirales, and realm Riboviria. Coronaviruses are enveloped viruses with a positive-sense single-stranded RNA genome and a nucleocapsid of helical symmetry. The genome size of coronaviruses ranges from approximately 26 to 32 kilobases, which is one of the largest among genome sizes amongst RNA viruses. Coronaviruses have characteristic club-shaped spike proteins (S) that project from their surface, which in electron micrographs create an image reminiscent of the solar corona, and hence their name.

Coronaviruses suitable for treatment by the disclosed methods may include, but are not limited to Human coronavirus 2229E, Human coronavirus NL63, Human coronavirus HKU1, Miniopterus bat coronavirus 1, Miniopterus bat coronavirus HKU8, Porcine epidemic diarrhea virus, Rhinolophus bat coronavirus HKU2, Scotophilus bat coronavirus 512, Betacoronavirus 1 (Bovine Coronavirus, Human coronavirus OC43), Human coronavirus HKU1, Murine coronavirus, Pipistrellus bat coronavirus HKU5, Rousettus bat coronavirus HKU9, severe acute respiratory syndrome-related coronavirus (SARS-CoV, SARS-CoV-2), Tylonycteris bat coronavirus HKU4, Middle East respiratory syndrome-related coronavirus, Hedgehog coronavirus 1 (EriCoV), Beluga whale coronavirus SW1, Infectious bronchitis virus, Bulbul coronavirus HKU11, and Porcine coronavirus HKU15. In particular, the disclosed methods may be practiced in order to treat infection with severe acute respiratory syndrome coronavirus 2 (SARS-CoV-2), which is the virus strain that causes coronavirus disease 2019 (COVID-19), a respiratory illness. SARS-CoV-2 utilizes the human angiotensin converting enzyme 2 cell surface protein to bind and target cells for infection via the spike protein (S).

In the disclosed methods, the disclosed compound have antiviral activity against an enveloped virus. In some embodiments, the disclosed compounds have antiviral activity against an enveloped virus selected from (i) inhibiting formation of an associative complex, (ii) disrupting formation of an associative complex, and (iii) both of (i) and (ii), optionally wherein the associative complex comprises an L-domain motif of the enveloped virus and at least one cellular polypeptide, or fragment thereof, capable of binding the L-domain motif of the enveloped virus. Preferably, the L-domain motif comprises at least one of a PY-motif and/or a PTAP-motif. Optionally, the L-domain motif comprises at least one amino acid sequence selected from the group consisting of SEQ ID NOS: 1-22 of U.S. Published Application No. 2019-0209589, the content of which is incorporated herein by reference in its entirety. Preferably, the at least one cellular polypeptide comprises an ESCRT complex protein. Optionally, the ESCRT complex protein comprises at least one member selected from a Nedd 4-related family peptide or a fragment thereof, TSG101 or a fragment thereof, and combinations thereof. Optionally, the ESCRT component protein comprises at least one amino acid sequence selected from the group consisting of SEQ ID NOS: 24, 25, 33, and 34 of U.S. Published Application No. 2019-0209589, the content of which is incorporated herein by reference in its entirety. The informal sequence listing that accompanies this application forms part of the description of the invention.

Inhibition of Viral Release from Infected Cells

The disclosed compounds or pharmaceutical compositions comprising the disclosed compounds may be administered in methods to inhibit the interaction of cellular proteins or fragments thereof with L domain-containing peptides of enveloped viruses. In particular, the methods and compositions may be utilized to inhibit the interaction of cellular proteins or fragments thereof, such as TSG101 or a fragment thereof or a Nedd 4-related family peptide or a fragment thereof, with L domain-containing peptides of enveloped viruses. The present inventors have determined that enveloped viruses use cellular pathways for mediating virus budding and that inhibiting these pathways results in significantly decreased rates of enveloped virus release from cell surfaces. The methods disclosed herein provide a robust, high-throughput approach to identify lead compounds having potent inhibitory effects on enveloped virus protein interactions with the components of these pathways and, thereby, virus particle release. Such methods offer a common mechanism to target a broad spectrum of viruses with a general anti-viral single therapeutic agent.

Enveloped viruses such as retroviruses and lentiviruses (e.g., avian sarcoma and leukosis virus (ASLV) and human immunodeficiency virus, type 1 (HIV-1)) include late assembly domains (“L-domains”) encoded within their Gag protein sequence that interact with cellular components of the endosomal sorting complex required for transport (“ESCRT”) machinery for virus budding and release from cells. The L-domains have been identified in a variety of enveloped viruses and families of enveloped viruses. A consensus subset of L-domain motifs that interact with the critical ESCRT-dependent processes that enveloped viruses use to bud from cell membranes is known. (See U.S. Publication Nos. 2014/0179637 and 2017/0095485, the contents of which are incorporated herein by reference in their entireties).

One of these L-domain motifs, termed the PTAP motif (for example, from HIV-1), interacts with the TSG101 protein that becomes recruited as part of the ESCRT complexes. Another of these L-domain motifs, termed PPPY motif (also referred to as the “PY motif” or the “PY L-domain motif” for example, from ASLV), interacts with the Nedd4 family of proteins that is also recruited by ESCRT-II-associated proteins or AIP1. While it is often the case that certain viruses have a viral protein might encode both types of L-domains, typically only one predominates in the viral budding process through interactions with ESCRT machinery. The inventors have devised novel, robust screening methods to identify compounds that interfere with the interaction between viral L-domains that include the PPPY motif or PTAP motif and ESCRT component, TSG101 or ESCRT-linked component, Nedd4 family proteins. These screening methods enable one to rapidly identify compounds that inhibit the interactions of both Nedd4 and TSG101 with the viral L-domain motifs, thereby providing a high-throughput strategy to obtain candidate lead compounds having utility as novel antiviral agents for inhibiting virus budding and release from infected cells.

Some candidate lead compounds can display potency at inhibiting only Nedd4-mediated ESCRT pathways or TSG101-mediated ESCRT pathways, thereby offering specific antiviral activity for one type of virus or virus family. Yet other candidate lead compounds can display potency at inhibiting both Nedd4-mediated ESCRT pathways and TSG101-mediated ESCRT pathways, thereby offering broad-spectrum antiviral activity to a plurality of diverse enveloped virus families using a common ESCRT pathway. Thus, the screening methods disclosed herein contemplate identification of compounds having either narrow-spectrum antiviral effects or broad-spectrum antiviral effects. Methods for identifying compounds that inhibit Nedd4-mediated ESCRT pathways and TSG101-mediated ESCRT pathways have been described. (See U.S. Publication Nos. 2014/0179637 and 2017/0095485, the contents of which are incorporated herein by reference in their entireties).

The compounds utilized in the treatment methods disclosed herein may exhibit one or more biological activities. The disclosed compounds may function to inhibit the release of enveloped viruses from infected cells. In some embodiments, the disclosed compounds inhibit release of enveloped viruses from infected cells by at least 50%, 60%, 70%, 80%, 90%, 95%, 96%, 97%, 98%, or 99% at a concentration of less than about 100 μM, 50 μM, 10 μM, 1 μM, 0.1 μM, 0.05 μM, 0.01 μM, 0.005 μM, 0.001 μM, or less, relative to a control. Preferably, the disclosed compounds are not toxic and/or do not inhibit the growth of cells (preferably by not more than 50%, 40%, 30%, 20%, 10%, 5%, 4%, 3%, 2% or less) at a concentration of greater than about 0.001 μM, 0.005 μM, 0.01 μM, 0.5 μM, 0.1 μM, 1.0 μM, 10 μM, and 100 μM or higher. Concentration ranges of the disclosed compounds for use in the disclosed methods also are contemplated herein, for example, a concentration range bounded by end-point concentrations selected from 0.001 μM, 0.005 μM, 0.01 μM, 0.5 μM, 0.1 μM, 1.0 μM, 10 μM, and 100 μM.

The compound utilized in the treatment methods disclosed herein may bind to one or more cellular proteins in order to inhibit viral release from infected cells. In some embodiments, the compounds utilized in the disclosed treatment methods may bind covalently to the one or more cellular proteins (e.g. TSG101).

FTS Assay for Detecting Direct Binding Interactions Between Test Compounds and ESCRT Component Proteins

The disclosed compounds or pharmaceutical compositions comprising the disclosed compounds may bind to one or more cellular proteins in order to inhibit viral release from infected cells. In some embodiments, the compounds utilized in the disclosed treatment methods may bind covalently and/or noncovalently to the one or more cellular ESCRT component proteins (e.g. TSG101 and/or Nedd4 proteins). Methods for detecting direct binding interaction between test compounds and ESCRT component proteins have been described an include fluorescence-based thermal shift (FTS) assays. (See U.S. Publication Nos. 2014/0179637 and 2017/0095485, the contents of which are incorporated herein by reference in their entireties).

In the disclosed methods and compositions, the therapeutic agent utilized in the disclosed methods and compositions may be a compound (or a small molecule) that binds to TSG101. In some embodiments, the therapeutic agent is a compound that binds to TSG101 with a dissociation constant (K_{d}(TSG101)) of less than about 10 μM, 5 μM, 2 μM, 1 μM, 0.5 μM, 0.2 μM, 0.1 μM, 0.05 μM, 0.02 μM, or 0.01 μM.

In the disclosed methods and compositions, the therapeutic agent utilized in the disclosed methods and compositions may be a compound (or a small molecule) that binds to Nedd4. In some embodiments, the therapeutic agent is a compound that binds to Nedd4 with a dissociation constant (K_{d}(Nedd4)) of less than about 10 μM, 5 μM, 2 μM, 1 μM, 0.5 μM, 0.2 μM, 0.1 μM, 0.05 μM, 0.02 μM, or 0.01 μM.

In some embodiments of the disclosed methods and compositions, the compound does not bind to H+/K+ ATPase of gastric parietal cells. If the compound binds to H+/K+ ATPase, preferably the compound binds to H+/K+ ATPase with a dissociation constant (K_{d}(H+/K+ ATPase)) greater than about 10 μM, 20 μM, 50 μM, 100 μM, 200 μM, 500 μM, or 1000 μM. In some embodiments, where the compound binds to TSG101 and/or Nedd4 and to H+/K+ ATPase the ratio K_{d}(TSG101)/K_{d}(H+/K+ ATPase) and/or K_{d}(Nedd4)/K_{d}(H+/K+ ATPase) is greater than about 10, 50, 100, 500, 1000, 5000, 10000, or higher. Preferably, the compound does not bind to H+/K+ ATPase and/or does not inactivate H+/K+ ATPase if the compounds binds.

The fluorescence-based thermal shift assay is based on the observation that a protein unfolds upon heating, exposing the hydrophobic residues within its tertiary structure. The unfolding temperature (Tₘ) is determined by the protein's primary sequence and solution environment. The FTS assay uses a fluorescent dye sensitive to a hydrophobic environment to probe protein stability and its modulation by small molecule ligands. The dye has a low fluorescence quantum yield when in a polar environment. Once in contact with the hydrophobic core normally buried within a folded protein that has become exposed during the thermal unfolding (melting) process, the quantum yield of the dye increases, thus providing a reporting signal. Furthermore, a protein's stability can be affected by ligand binding, resulting in an increase or decrease in its melting temperature. FTS assay uses the Tₘ shift upon binding of a ligand to identify hit compounds for drug discovery.

In Vivo Screening Methods-Based Molecular Genetic Assays, Cell-Based VLP Production Assays and Whole-Virus Replication Assays

Candidate compounds having an inhibitory effect of fluorescence have also been evaluated for their ability to interfere with normal cellular physiology and growth by, for example, determining cytotoxicity profiles of the compounds as a function of dose response and incubation time with the cells. One advantage of the in vivo assay is that it provides additional opportunities to survey test compounds that otherwise might not be possible with the aforementioned biochemical assays (for example, with assays involving certain ESCRT component polypeptides having limited solubility in vitro). Other further advantages of in vivo assays of this sort is that they can provide a useful model for studying compound transport and clearance in cells as would be important for determining ADME profiles (for examples, bioactivity, bioavailability, bio-inactivation, among others) at a cellular level, as well as provide additional confirmatory evidence of the biological potency of the compounds in a more meaningful, biological context.

For candidate lead compounds identified through one or more of the aforementioned screening methods, biological assays have been established to evaluate the specific antiviral inhibitory effects the compounds have on virus budding and release. In one assay, virus like particle (“VLP”) production can be evaluated as a function of test compound dose. Follow-up experiments well within the skilled artisan's grasp include evaluating other aspects of viral replication, as monitored by standard biochemical assays (PCR, RT-PCR, western blot methods and the like) as well as cell toxicity effects. These assays and other aspects are described in detail in the Examples or are otherwise well understood in the art. VLP production assays have provided evidence of candidate lead compounds showing antiviral inhibitory effect on virus particle release as a function of dose, experiments then can proceed to demonstrate the antiviral effect in whole virus replication assays.

The aforementioned in vivo and in vitro screening methods can be combined either in series or in parallel (and in any order) to identify compounds having either narrow-spectrum activity against a few viruses or broad-spectrum antiviral activity against many different viruses. In this manner, different antiviral compounds can be discerned having discrete types of inhibitory activity. Further, one can identify gradients of antiviral potency across entire classes of viruses by evaluating the dose response profiles in a combination of biochemical and biological assays with different virus families having different viral L-domain motifs, as described herein. Moreover, combinations of compounds have TSG101-specific inhibitory activity and Nedd4 family-specific inhibitory activity can be tested against virus infection to determine whether the drug combinations block virus access to the ESCRT-complex dependent pathways are blocked for enveloped virus release.

These approaches have clear utility for two simple reasons. First, L-domains encoding the aforementioned PY motifs and PTAP motifs can be found with viral proteins for single virus families. Thus, viruses having both types of L-domains can potentially utilize both pathways mediated by Nedd 4 and TSG101. Second, the L-domains used by viruses are interchangeable. Thus, there is a need for compounds to disrupt both interactions between viral L-domains with the two different pathways mediated by Nedd 4 and TSG101, wherein virus budding and release can occur from different cellular membranes.

The identified prazole compound inhibitors have utility as antiviral therapeutic agents. The therapy is a post infection treatment that will slow down the spread of virus by preventing particles from releasing from infected cell surfaces. The accumulation of particles will enhance detection by the immune system, which will clear the infection. The human body already has an innate immunity response that targets the release of virus particles late in infection. Thus, the above approach has viability because it will complement the natural immunity mechanism.

### EXAMPLES

The following Examples are illustrative and should not be interpreted to limit the scope of the claimed subject matter.

### Example 4

Reference is made to Leis et al., “Ilaprazole and other novel prazole-based compounds that bind Tsg101 inhibit viral budding of HSV-1/2 and HIV from cells,” J. Virol., 2021 Mar. 17; JVI.00190-21.doi: 10.1128/JVI.00190-21, the content of which is incorporated herein by reference in its entirety.

Abstract

In many enveloped virus families, including HIV and HSV, a crucial, yet unexploited, step in the viral life cycle is releasing particles from the infected cell membranes. This release process is mediated by host ESCRT complex proteins, which are recruited by viral structural proteins and provides the mechanical means for membrane scission and subsequent viral budding. The prazole drug, tenatoprazole, was previously shown to bind to ESCRT complex member Tsg101 and to quantitatively block the release of infectious HIV-1 from cells in culture. In this report we show that tenatoprazole and a related prazole drug, ilaprazole, effectively block infectious Herpes Simplex Virus (HSV)-1/2 release from Vero cells in culture. By electron microscopy, we found that both prazole drugs block the transit of HSV particles through the cell nuclear membrane resulting in their accumulation in the nucleus. Ilaprazole also quantitatively blocks the release of HIV-1 from 293T cells with an EC₅₀ of 0.8-1.2 μM, which is much more potent than tenatoprazole. Our results indicate that prazole-based compounds may represent a class of drugs with potential to be broad-spectrum antiviral agents against multiple enveloped viruses, by interrupting cellular Tsg101 interaction with maturing virus, thus blocking the budding process that releases particles from the cell.

Importance

These results provide the basis for the development of drugs that target enveloped virus budding that can be used ultimately to control multiple virus infections in humans.

### INTRODUCTION

The advent of antibiotics had a major impact on controlling bacterial infections in patients worldwide, with a single drug being used to treat multiple infections. Unfortunately, antivirals have not had the same success. There are many contributing factors to this shortcoming, foremost the fact that few mechanisms are shared by different viruses, which limits targets for a broad-spectrum antiviral. Consequently, approved antivirals generally act against individual rather than groups of viruses, limiting a single drug's potential.

Enveloped viruses bud from the host cell membranes and use the acquired lipid layer as a protective coat that also contains the glycoproteins required for infection of other cells. Enveloped viruses do not encode the machinery needed for budding and must recruit host-cell proteins to bud from cells. In HIV, ESCRT proteins are recruited to virus budding complexes through an interaction between the L-34 domain (PT\SAP motif) in virus structural proteins (3-7) with cellular protein Tsg101 (Tumor susceptibility gene 101), a homolog of the E2 ubiquitin conjugating enzyme and a member of the ESCRT-I complex (6, 8-11). Tsg101 recruits the cellular ESCRT-III complex, which provides the mechanical means for viruses to passage through cell membranes to be released from cells (10, 12-19). In contrast to HIV, herpes simplex virus (HSV) assembles particles in the nucleus and must passage through the nuclear membrane into the cytoplasm where it exchanges membranes to become infectious and then is released from the cell membrane. The ESCRT proteins are required for this passage (20, 21, 29). Thus, virus budding may present a common target for treating multiple virus infections.

In support of targeting this pathway, a recent seminal discovery in our lab established that an interferon-induced protein, Interferon Stimulated Gene 15 (ISG15), specifically targets the ESCRT-III proteins in budding complexes to block the release of viruses (1, 22-24). This indicates that the human immune system evolved to target the ESCRT pathway to control viral infections and supports that this is a natural target. Another group identified single-nucleotide polymorphic sites in the 5′ region of Tsg101, located at positions-183 and +181 relative to the translation start signal, which affect the rate of AIDS progression among Caucasians (25). These data support the hypothesis that variation in Tsg101 affects efficiency of Tsg101-mediated release of viral particles from infected cells, altering plasma viral load levels and subsequent disease progression. Taken together, these investigations indicate that Tsg101 and ESCRT proteins present a natural antiviral target.

Currently the prazole family of drugs is best known for their role as proton pump inhibitors (PPIs) and a few, namely omeprazole (Prilosec), esomeprazole (Nexium) and ilaprazole (Adiza, Noltec, Yi Li An), are marketed to control symptoms of gastroesophageal reflux disease (GERD) in either the US or abroad. PPIs form a covalent bond with the active site of proton pumps, inhibiting their ability to acidify the stomach and reducing symptoms associated with over-acidification (26). Recent reports indicate that drugs from the prazole family, including tenatoprazole and esomeprazole, form a disulfide linkage to Tsg101, which results in blocking the release of HIV-1 from cells in culture (5).

In the present manuscript, we demonstrate that multiple prazole drugs block the budding of HSV-1 64 and HSV-2 from Vero cells in culture, strengthening the case for the broad-spectrum potential of this mechanism/drugs. Most notably, we identified one prazole drug, ilaprazole, which blocks the release of both HIV-1 and HSV-1/2 from cells at an efficiency more potent than reported for tenatoprazole. Ilaprazole acts in the low μM range without detectable cell toxicity at inhibitory concentrations. To further define the mechanism of action of these prazole drugs on HSV infections, we identified the site of blockage of herpesvirus release, which appears to be different from HIV-1. While the blockage to HIV-1 particle release is at the outer cell membrane (5), the prazole drugs appear to first block the passage of the herpesvirus through the nuclear membrane. This prevent s particles being released into the cytosol, where maturation of their envelope membrane occurs to produce infectious virus and where they bud from the cell. With the prazole-based inhibitors being effective in both HIV and HSV, targeting Tsg101 could lead to a broad-spectrum antiviral therapy.

Results

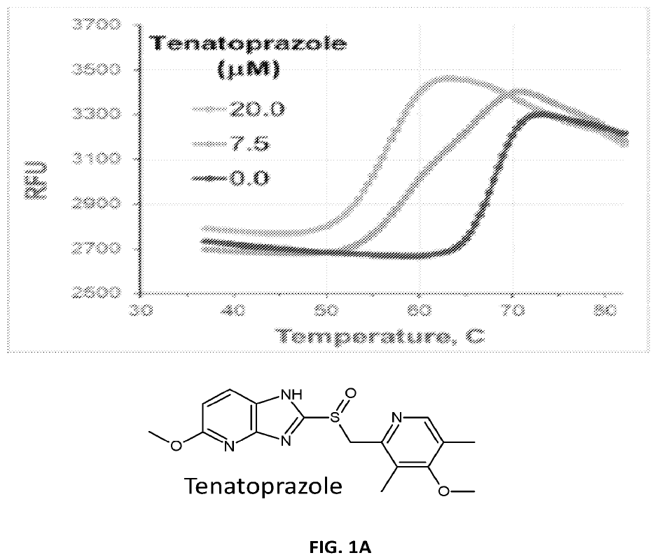

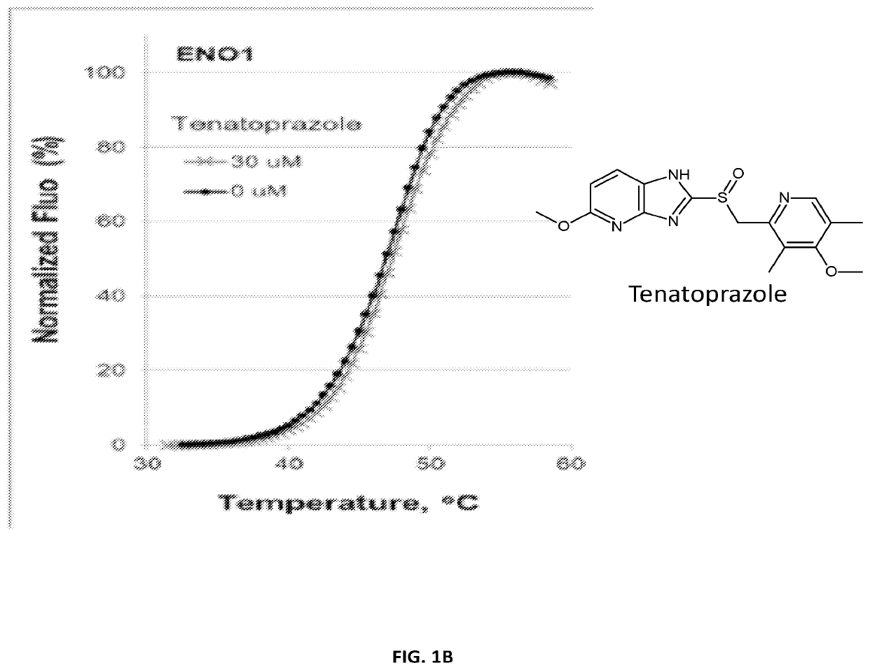

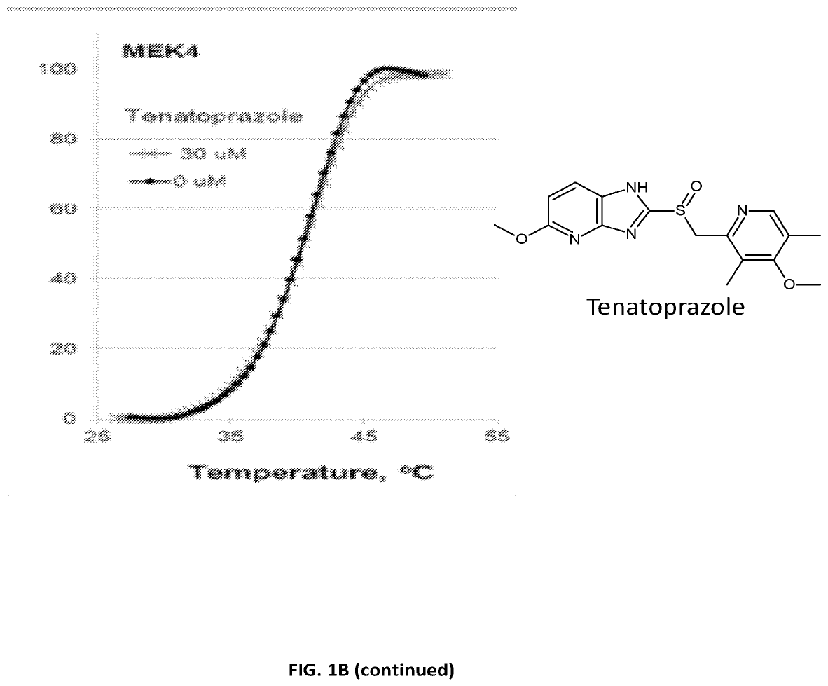

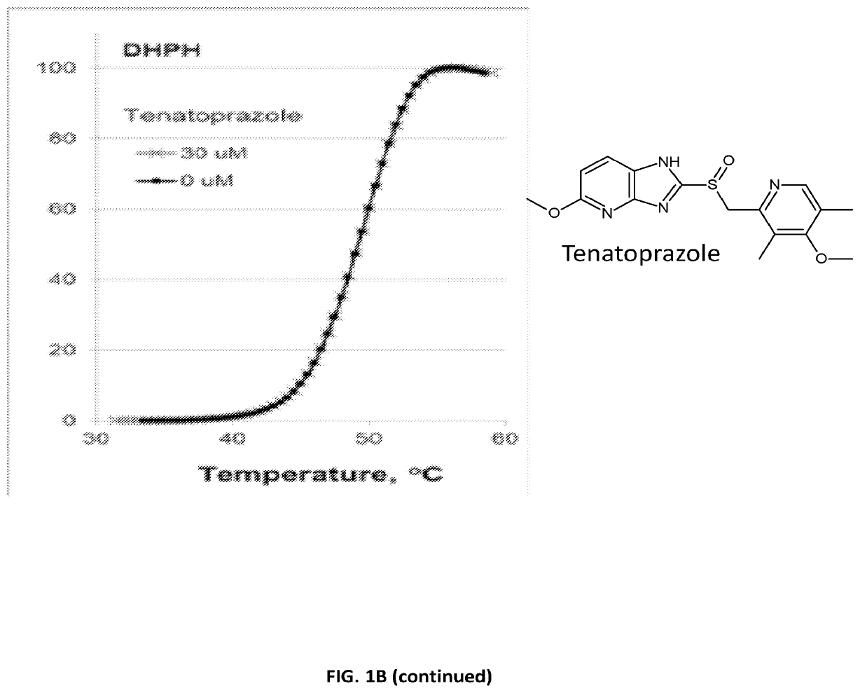

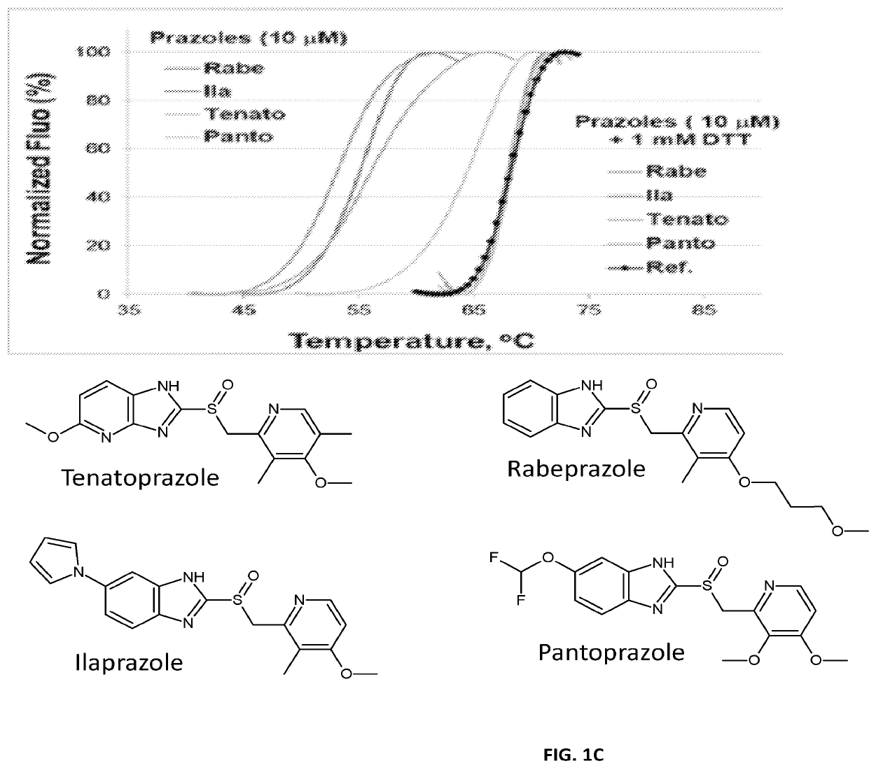

Identification of prazole compounds that bind the UEV ubiquitin-binding domain of Tsg101. We screened chemical compounds using a fluorescence thermal shift (FTS) assay (27, 28) to identify small molecules that bind directly to a truncated form of Tsg101 (amino acids 1-145) which contains the Ubiquitin E2 variant (UEV) ubiquitin-binding domain (FIG. 1A, 1B, 1C). The UEV, which contains the PT/SAP binding domain in addition to the ubiquitin-binding domain, provides chaperone functions to HIV-1 Gag that is independent of its interaction with the PS/TAP motif, and contains the prazole binding site (5). This truncated Tsg101, called Tsg101-UEV, was used because full-length Tsg101 has significant solubility issues in aqueous solution. Tsg101 is an adaptor protein and thus lacks a readily deployable functional assay, making FTS a tractable approach to identify interacting compounds. FTS monitors protein thermal denaturation using SYPRO® Orange, a dye which fluoresces when bound to hydrophobic surfaces, which allows monitoring of the changes in hydrophobic surface exposure during protein denaturation (27). Since ligand binding affects protein thermal stability, it can be detected through modulation of protein thermal denaturation (melting) as a shift in melting temperature (Tₘ). Tsg101-UEV has a well-defined melting curve suitable for FTS. We used the FTS assay to identify compounds that bind to Tsg101-UEV.

We compared thermal denaturation profile for Tsg101-UEV in the presence and absence of tenatoprazole and found that it destabilizes the native protein structure, indicating that it binds Tsg101-UEV (FIG. 1A). We also tested tenatoprazole against proteins unrelated to Tsg101, including DHPH, ENO1, MEK4, and did not observe a Tₘ shift, indicating that the Tₘ shift of Tsg101-UEV was due to specific interaction of the prazole compound (FIG. 1). This specific binding is consistent with a previous NMR structure in which tenatoprazole forms a covalent disulfide bond to Cys73 in the UEV domain of the protein (5). This disulfide bond formation can be prevented by including the reducing agent DTT in the assay (FIG. 1C). The addition of DTT abolished the Tsg101-UEV Tₘ shift caused by the prazoles. Therefore, the addition of DTT to the FTS assay is a facile means to ascertain if prazole analogs interact with Tsg101-UEV in a covalent manner.

Tenatoprazole inhibits herpesvirus release from Vero cells. Tenatoprazole and esomeprazole were shown to quantitatively inhibit the release of infectious HIV-1 from 293T cells in culture, and it was suggested that these effects may be mediated via changes in viral interaction with Tsg101, a key component of the cellular ESCRT complex (5, 29). Given multiple reports suggesting that herpesviruses also use cellular ESCRT proteins in their replication process (20, 21) we tested if the Tsg101-binding prazole drugs, which blocked budding of HIV-1, would also block the release of herpesviruses from cells.

We infected Vero cells with HSV-1 and HSV-2 for two hours at a multiplicity of infection (MOI in 113 pfu\cell) of 0.1 to assay the antiviral activity of tenatoprazole. Following infection, cells were treated with different concentrations of tenatoprazole. After 24 or 48 h the media fractions were collected and released virus titers were determined by standard plaque assays (30). Tenatoprazole caused a 3-log drop of HSV-1 and 4-5 log drop of HSV-2 in released infectious virus from Vero cells at 24 hours after infection in a dose dependent manner (Table 1, columns 2 and 3) with calculated EC50's ranging from 48-80 μM. Similar results were obtained at 48 h after infection (Table 1, columns 5 and 6). Total virus titer was also determined to differentiate between virus released into the media and infectious particles present in cell lysate. Total infectious virus particles were reduced by tenatoprazole, but not as strongly as virus released into the media (Table 1, compare column s 3 and 4). The concentrations of tenatoprazole that blocked virus release were nontoxic to Vero cells as determined by a 96® AQueous One Solution cell proliferation assay reagent (Table 1, column 7). Taken together, tenatoprazole inhibited levels of both released and infectious virus particles without affecting cell viability at effective concentrations.

TABLE 1
Effect of Tenatoprazole on HSV-1 and HSV-2 release from Vero cells. Tenatoprazole was incubated with Vero
cells infected with HSV-1 or HSV-2 at a range of concentrations. The virus released into the media fraction
at stated times was determined as described in Materials and Methods. Total virus is the amount of virus
released from cells plus virus inside of the cells. Viability of Vero cells incubated with increased concentration
of tenatoprazole was determined using the 96 ® AQueous One Solution cell proliferation assay reagent
as described in Materials and Methods. Total titer for HSV -1 was not included. Duplicate plaque assays
of 10 -fold serial dilutions were determined with an average of less than 13% difference in the number
of plaques counted. The 24 h, 48 h data were repeated 6 times each. The total virus was repeated twice.
The data presented is the average of 2 experiments where the titers varied between 5 to 20%.

| | | | Total Titer HSV-2 | | | |
|---|---|---|---|---|---|---|
| Tenatoprazole | Titer of HSV-1 | Titer of HSV-2 | Media + Cell | Titer of HSV-1 | Titer of HSV-2 | Viability of OD |
| (μM) | Media, 24 h | Media, 24 h | Lysate, 24 h | Media, 48 H | Media 48 h | 490 nm, 24 h |
| 0 | 2.50E+05 | 2.80E+05 | 4.70E+07 | 8.0E+07 | 8.50E+06 | 1.694 |
| 52 | 2.90E+05 | 6.50E+04 | 4.00E+06 | 1.4E+07 | 1.30E+06 | 1.724 |
| 60 | N.D. | 1.00E+03 | 2.30E+04 | N.D. | 5.60E+04 | 1.759 |
| 79 | 1.30E+05 | 2.50E+02 | 1.50E+03 | 8.0E+06 | 4.80E+03 | 1.742 |
| 105 | 5.40E+04 | 0.00E+00 | 6.00E+02 | 5.3E+05 | 1.80E+02 | 1.777 |
| 131 | 2.40E+03 | 0.00E+00 | 3.00E+02 | 3.5E+04 | N.D. | 1.714 |
| 157 | 1.30E+02 | N.D. | N.D. | 1.0E+02 | N.D. | N.D. |
| 200 | N.D. | N.D. | N.D. | N.D. | N.D. | 0.872 |

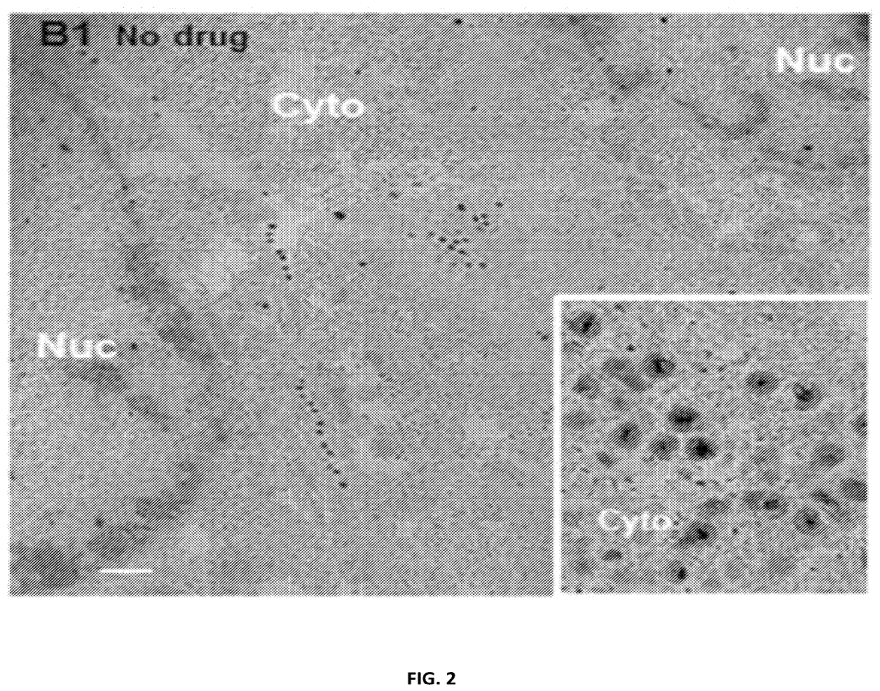

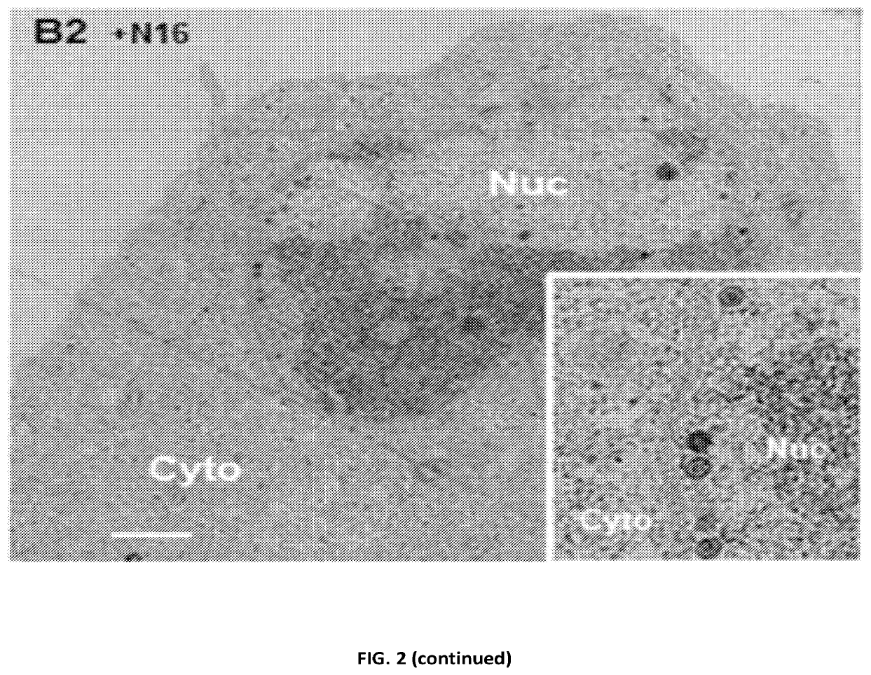

Cellular location of tenatoprazole inhibition. We next imaged herpesvirus infected-Vero cells using transmission electron microscopy to determine the site of inhibition of release of virus and whether it was similar to observations of HIV-1 release from 293T cells. Vero cells grown on glass cover slips were infected with HSV-2 at MOI of 0.1 pfu/cell for 2 h and then treated for 24 h with 105 μM tenatoprazole or vehicle control. Using electron microscopy, we examined eighty cells with virus particles, and representative images are shown in FIG. 2. In the no drug control, virus particles were in both the nucleus and cytoplasm near the cell surface (FIG. 2). In the tenatoprazole-treated cells the cytosol of all of the intact cells was largely devoid of virus particles (FIG. 2). Instead, we observed large pockets of granular material accumulated in the nucleus and immature virus particles inside the nucleus and lining the inside of the nuclear membrane. This result suggests that tenatoprazole blocks the passage of herpesvirus particles through the nuclear membrane, in contrast to the report of Pawliczek and Crump (31). This result also differs from that observed with HIV-1. Because tenatoprazole binds Tsg101, it suggests that the ESCRT-I protein complex is involved in transport of HSV-2 through the nuclear membrane and/or particle assembly.

Identification of potent prazole-based inhibitors. Despite the lack of cell toxicity signal at effective tenatoprazole concentrations, the effective concentration is too high for use as a clinical therapy. Therefore, more potent analogs are required to explore antiviral therapeutic potential. We set out to identify and test other analogs which were more potent prazole analogs. We began by searching PubChem for analogs of tenatoprazole. We identified and obtained a dozen such compounds from commercial sources and prioritized these for testing based on structural similarities around the sites where tenatoprazole covalently linked to Cys73 of Tsg101. To this end, tenatoprazole, lansoprazole, rabeprazole, dexlansoprazole, pantoprazole, esomeprazole, 4-desmethoxy-omeprazole (an omeprazole analog, 5-methoxy-2[[(3,5-dimethyl-4-methoxy-pridin-2-yl-N-oxide)methyl]sulfinyl]-1H-benzimidazole; labelled O-Omeprazole), omeprazole, and ilaprazole were assessed in the FTS assay for their ability to change the Tₘ of Tsg101-UEV as described above (data not shown).

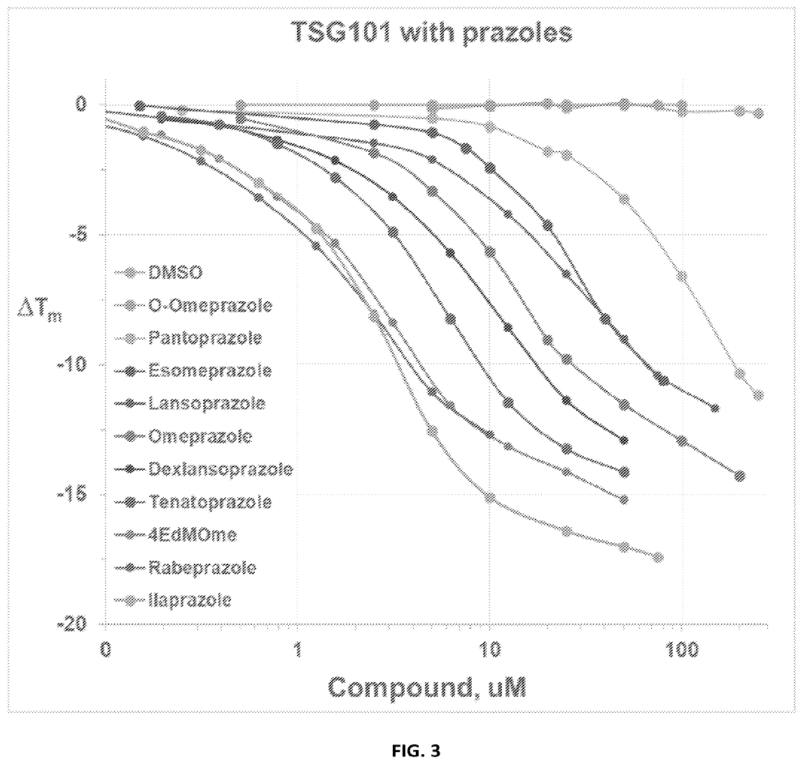

We determined the dose response plots of Tsg101 melting temperature shifts caused by these prazole compounds binding to Tsg101 (1-145) (FIG. 3). O-omeprazole is the only compound predicted not to form the covalent bond with Tsg101, since it has an oxygen linked to a ring nitrogen that is normally a hydrogen in the active prazoles (Table 2, right column). As expected, O-omeprazole did not cause a detectable thermal shift (FIG. 3). The smallest thermal shift was observed with pantoprazole and the largest thermal shift was observed with ilaprazole. Ilaprazole's ability to cause a thermal shift with Tsg101 was blocked by the addition of DTT (FIG. 1C), consistent with the idea that the compound forms a disulfide linkage to Tsg101.

Next, we tested the anti-herpesvirus activity of these prazole compounds (Table 2). To examine the effects of the compounds on the release of HSV-2 from Vero cells, we infected the cells with virus two hours prior to treatment with media containing different concentrations of compound. We incubated the cells for 24 or 48 hours and then collected the cell media fractions. Several of the analogs were inactive, including O-Omeprazole, pantoprazole, and rabeprazole. We identified a number of active compounds, in which there was a 10-fold spread of inhibition activity against HSV-2, ranging from an EC₅₀ of 140 μM (for esomeprazole) to 3-9 μM (for ilaprazole). Thus, we identified prazole analogs that are more potent than tenatoprazole.

TABLE 2
Effect of commercial prazole analogs to inhibit the release of HSV-2
from Vero cells. Different concentrations of the listed prazole drugs were incubated with
HSV-2 infected Vero cells for 24 hours and then virus released into the media was quantified by
plaque assays. Data presented includes the EC50 value (concentration at which virus release is
inhibited by 50%). Methods are as described in the legend of Table 1.

| | EC₅₀ (μM) Inhibition of HSV-2 | |
|---|---|---|
| Prazole Compound | Budding at 24 h | Structure |
| O-omeprazole | — | 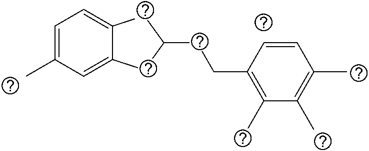 |
| Pantoprazole | — | 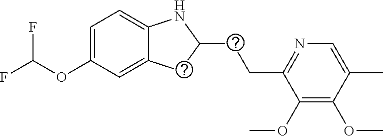 [SMILES: COc1c(C)cnc(CCC2Cc3cc(OC(F)F)ccc3N2)c1OC] |
| Esomeprazole | 140 | 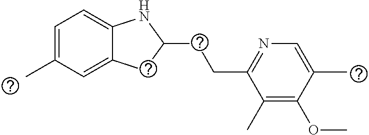 |
| Lansoprazole | 84 | 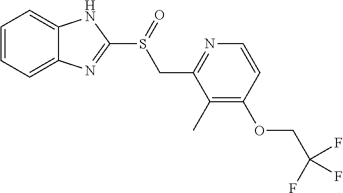 [SMILES: Cc1c(OCC(F)(F)F)ccnc1CS(=O)c1nc2ccccc2[nH]1] |
| Omeprazole | 78 | 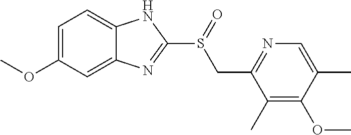 |
| Dexlansoprazole | 76 | 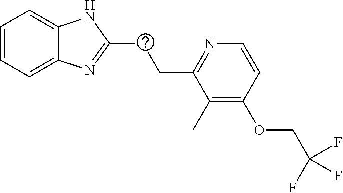 [SMILES: Cc1c(OCC(F)(F)F)ccnc1CCc1nc2ccccc2[nH]1] |
| Tenatoprazole | 84 | 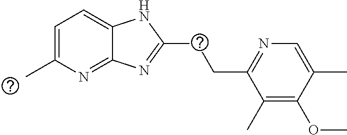 |
| 4-Desmethoxy-omeprazole | 52 | 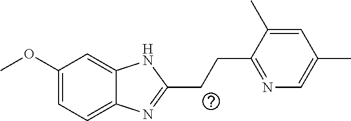 |
| Rabeprazole | — | 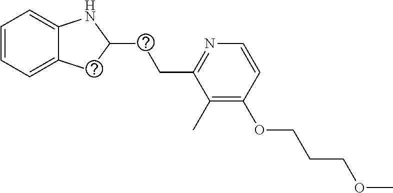 [SMILES: COCCCOc1ccnc(CCC2Cc3ccccc3N2)c1C] |
| llaprazole | 3-9 | 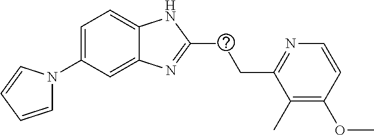 [SMILES: COc1ccnc(CCc2nc3cc(-n4cccc4)ccc3[nH]2)c1C] |

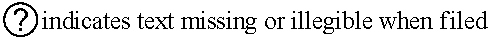

[symbol]

We provide the structures of prazole compounds tested in this analysis (Table 2, column 3). Of note, ilaprazole contains an additional ring structure compared to tenatoprazole that is predicted to lie in a solvent exposed area of the Tsg101 structure that may serve to strengthen the interaction with Tsg101. In examining the thermal shift capacity of the prazoles, we found that the larger the thermal shift the more potent antiviral activity associated with the compound. This correlation indicates that the FTS assay is useful in evaluating structure-activity-relationships (SAR) to inform the design of new compounds (FIG. 3, Table 2).

Antiviral activity of Ilaprazole on HSV-1 and HSV-2 in vitro. Based on these initial HSV-2 antiviral assay results, we selected ilaprazole for further antiviral profiling and tested it against HSV-1 (Table 3A, columns 2-5) and HSV-2 (Table 3A, columns 6-8). Ilaprazole was slightly more effective against HSV-1 than against HSV-2 with EC₅₀ calculations ranging from 3-9 μM. These results do not indicate if the observed lower EC₅₀ against HSV-1 compared to HSV-2 is significant or reflects differences between different viral isolates, since the two herpesviruses can be distinguished by sequence analysis and both types can cause oral and genital lesions. Ilaprazole's potency is an improvement over tenatoprazole, which inhibited in the high μM range (Table 1 & 3). Like tenatoprazole, ilaprazole caused a significant drop in total virus, again not as strong decrease as detected with virus released from cells. Additionally, ilaprazole was even more effective in inhibiting virus release at 72 h as at 24 h after a single application of the drug (72 h EC₅₀ 0.8-1.2 μM; compare Table 3A, columns 2 & 4). Significant inhibition was still observed at 4 and 5 days after a single application of the drug (data not shown). The inhibition caused by tenatoprazole against either virus began to fall off after 48 h (data not shown). We also tested for toxicity in the range of effective concentrations and did not observe cell toxicity using the 96® AQueous One Solution cell proliferation assay reagent and WST-1 reagent over a 24 h period (Table 3B). Thus, ilaprazole is more potent and has longer lasting effects than tenatoprazole.

Table 3A and 3B. Effect of Ilaprazole on release of HSV -1 and HSV -2 from Vero cells. Different
concentrations of ilaprazole were incubated for the times indicated with HSV -1 or HSV -2
infected cells similar to that described in the legend to Table 1. Virus titer released into
the media and total virus was determined. Viability of Vero cells incubated with increased
concentration of tenatoprazole was determined using the 96 ® AQueous One Solution
cell proliferation assay reagent as described in Materials and Methods. Data were analyzed
as described in legend to Table 1 and experiments were repeated 4 times each.
A

| | | | | Titer of | | Titer of | |
|---|---|---|---|---|---|---|---|
| | Titer of | Titer of | Total Titer | HSV-1 in | Titer of | HSV-2 in | Titer of |
| Ilaprazole | HSV-1 | HSV-1 | HSV-1 in | media + cell | HSV-2 | media + cell | HSV-2 in |
| (μM) | Media, 24 h | Media, 48 h | media, 72 h | lysate, 24 h | Media, 24 h | lysate, 24 h | media, 48 h |
| 0 | 3.00E+06 | 3.90E+07 | 1.00E+08 | 2.2E+08 | 2.80E+05 | 1.20E+07 | 1.00E+06 |
| 4.5 | 2.00E+06 | 2.40E+06 | 9.00E+05 | 7.0E+07 | 1.00E+05 | 3.60E+07 | 2.50E+05 |
| 9.0 | 7.50E+04 | 2.50E+05 | 2.20E+05 | 3.6E+06 | 5.00E+04 | 4.50E+06 | 7.50E+04 |
| 13.5 | 3.20E+04 | 2.00E+02 | 4.50E+02 | 3.8E+05 | 1.00E+04 | 4.30E+06 | 5.50E+04 |
| 18.0 | 6.00E+02 | 0.00E+00 | 1.00E+02 | 9.1E+03 | 1.50E+03 | 2.00E+05 | 1.50E+03 |
| 22.5 | 1.00E+02 | 0.00E+00 | N.D. | 7E+02 | 1.00E+02 | 1.50E+04 | 3.00E+02 |
| 54 | N.D. | N.D. | N.D. | N.D. | N.D. | N.D. | N.D. |
| 270 | N.D. | N.D. | N.D. | N.D. | N.D. | N.D. | N.D. |

B

| Ilaprazole | 96 ® Cell Viablility | WST-1 Cell Toxicity |
|---|---|---|
| (μM) | OD_{490 nm} 24 h | OD_{440-660 nm}, 24 h |
| 0 | 1.694 | 0.993 |
| 4.5 | 1.764 | 1.058 |
| 9.0 | 1.711 | 1.055 |
| 13.5 | 1.690 | 0.950 |
| 18.0 | 1.737 | N.D. |
| 27 | N.D. | 1.055 |
| 54 | 1.658 | N.D. |
| 270 | 0.466 | 0.423 |

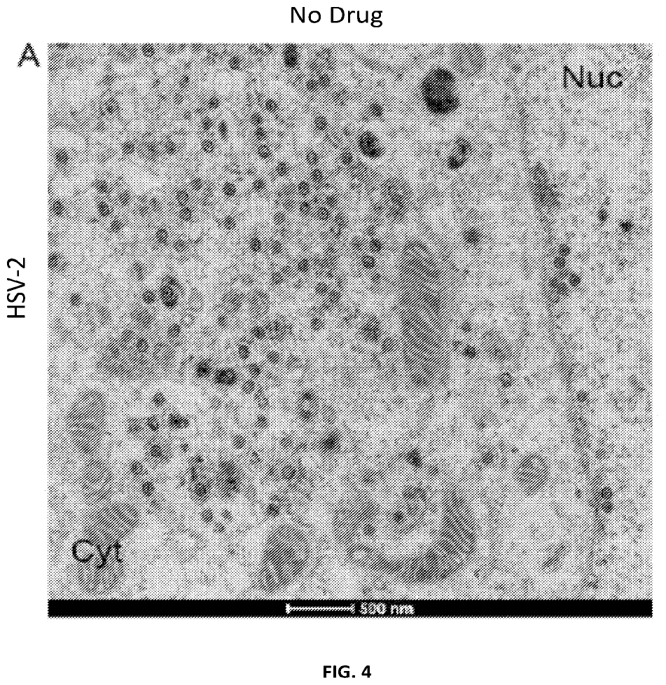

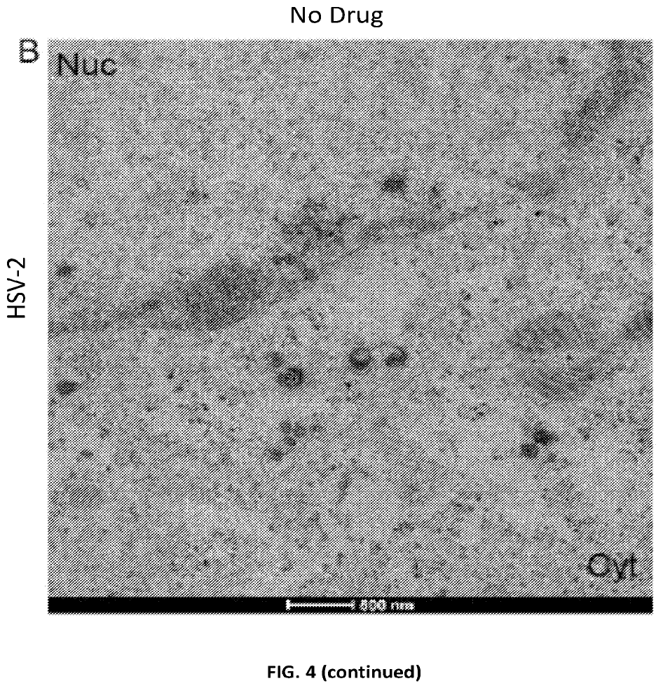

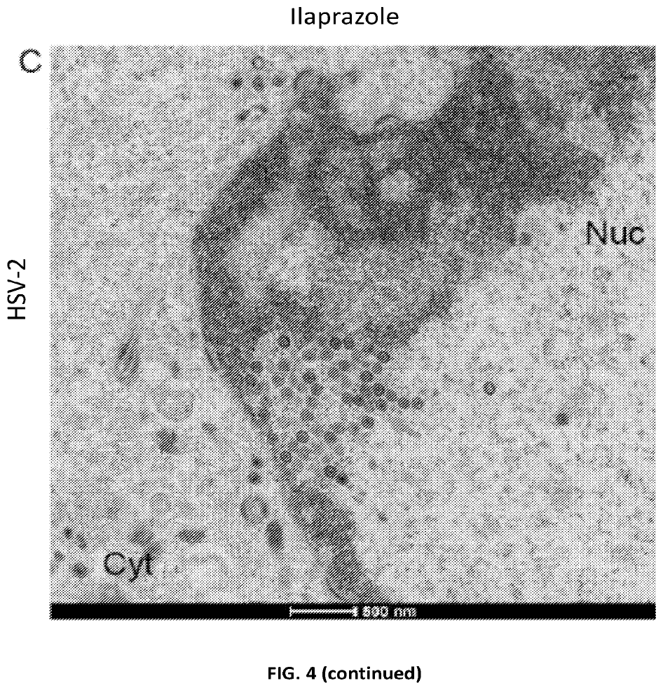

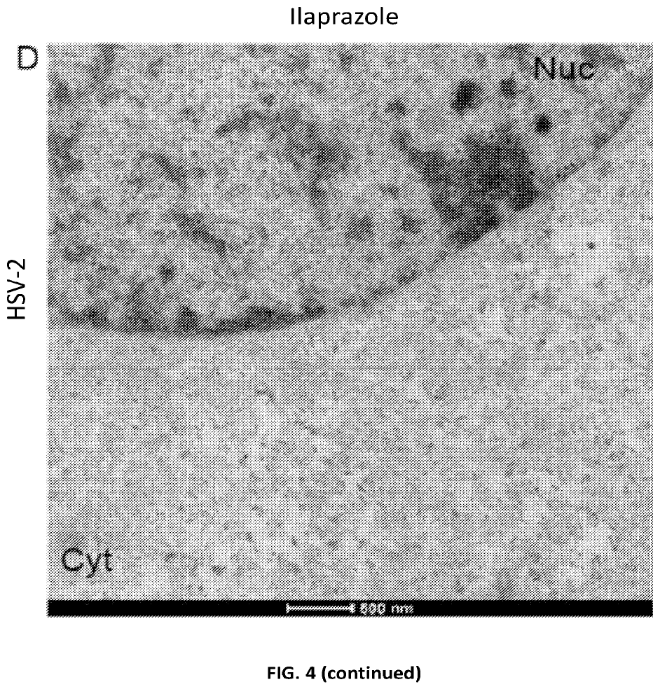

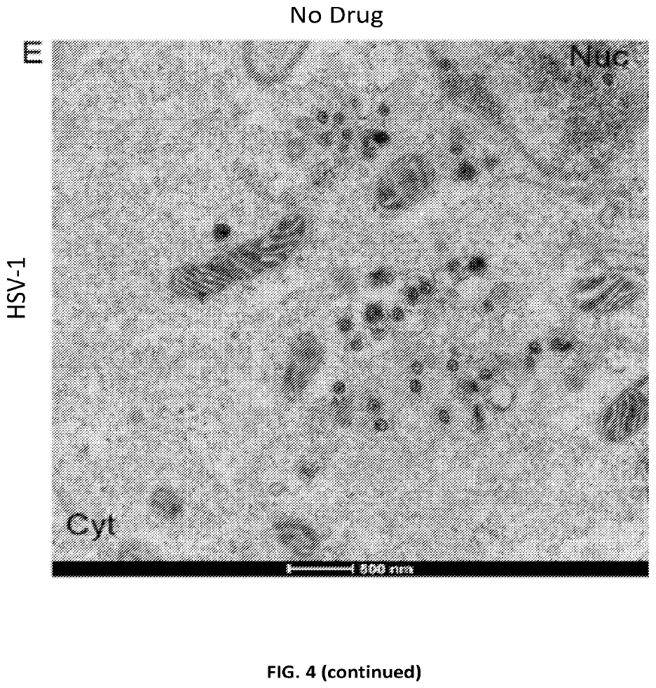

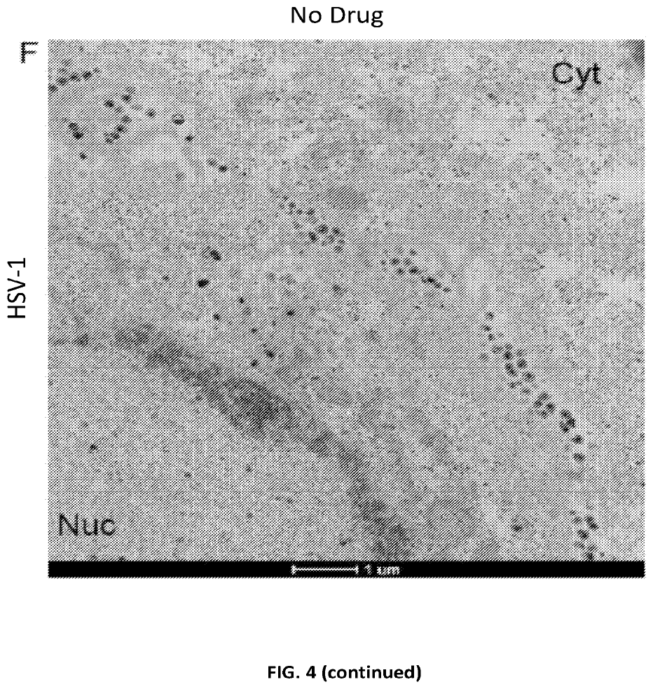

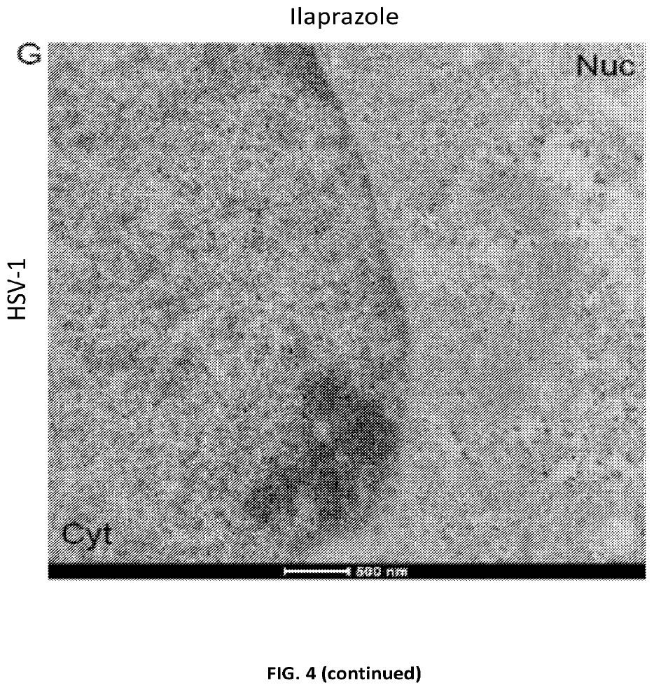

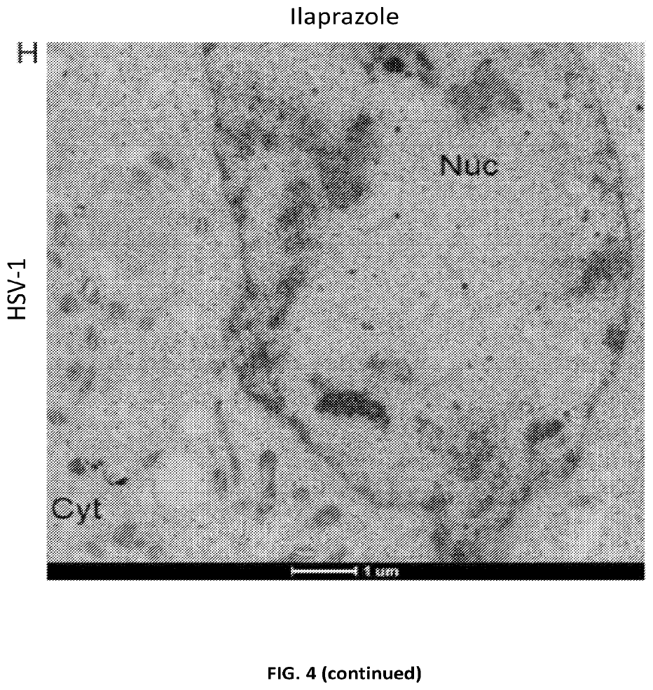

We next carried out a transmission electron microscopic examination of cells infected with HSV-2 at a MOI 0.1 in the presence and absence of 18 μM ilaprazole to determine if this drug causes the accumulation of virus particles in the nucleus of cells similar to tenatoprazole. Without drug, we observe particles in the cytoplasm and in the nucleus (FIGS. 4A & B), in the presence of drug little or no viral particles are found in the cytoplasm (FIGS. 4C & D). In both heavily infected cells (FIGS. 4A & C) and mildly infected cells (FIGS. 4B & D), treatment lead to particles being detected in the nucleus and arrayed along the nuclear membrane but lacking in the cytosol. This indicates that location of particles in the cell in the presence of drug is independent of the number of particles observed. Similar results were obtained with HSV-1 infected cells (FIG. 4E-H). Particles are seen in both the cytoplasm and nucleus in the absence of drug and just in the nucleus in the presence of drug. These results are similar to the effect of tenatoprazole on HSV-2 infected cells (FIG. 2). The lower total infectious virus detected in Table 3 is consistent with blockage of the virus passaging out of the nucleus into the cytoplasm where membranes are exchanged and virus becomes infectious.

Effect of Ilaprazole on release of HIV-1 in vitro. To establish the broad-spectrum potential of ilaprazole, we tested whether ilaprazole would inhibit the release of HIV-1 from 293T cells. To this end, cells were transfected with pR9-HIV-1Ba-L plasmid and release of virus into the media fraction was detected by monitoring the capsid (CA) protein (p24) via enzyme linked immunosorbent assay analysis. The drug was tested at concentrations between 0 and 240 μM and the effect of the drug on release of virus assessed (Table 4, column 2). Ilaprazole was effective at inhibiting the release of HIV-1 from cells with a calculated EC₅₀ of 1 μM or less as described in Materials and Methods. We did not detect toxicity to the cells at the drug concentrations that inhibited the release of HIV-1 over the course of these experiments. Thus, ilaprazole has antiviral activity against HSV-1, HSV-2, and HIV-1, demonstrating its potential as a broad-spectrum antiviral.

TABLE 4
Effect of ilaprazole and novel analogs on release of HIV from
293T cells. Different concentrations of ilaprazole were incubated
with HIV-1 plasmid transfected cells as described in Materials
and Methods. Virus titer released into the media was determined
by monitoring p24 levels 24 h post-infection using a fluorescent
labeled antibody. Each experiment was repeated 4 times and
the average p24 CA pg/ml presented. Cell toxicity experiments
were repeated 2 times each.

| | | | 96 ® Cell |
|---|---|---|---|
| | | | Viability |
| Ilaprazole | p24 pg/ml | Ilaprazole | OD_{490 nm}, |
| μM | HIV | μM | 24 h |
| 0 | 7021 | 0 | 1.694 |
| 0.5 | 4063 | 1.8 | 1.672 |
| 1 | 3518 | 3.6 | 1.576 |
| 2 | 1652 | 7.2 | 1.684 |
| 5 | 274 | 10.8 | 1.650 |
| 10 | 178 | 13.5 | 1.693 |

Discussion

We are developing a novel strategy to treat viral infections affecting humans by disrupting a common mechanism used by many enveloped viruses to bud from cells. Viral budding Inhibitors (VBI) have the potential to be broad-spectrum antiviral therapeutics, potentially being effective against herpesvirus (29, 31-35), retro/lenti- (5, 29), arena- (LFV, LCMV) (36, 37), flavi- (HCV) (38, 39), filo- (Ebola, MarV) (40-47), hepadna- (HBV) (48), some paramyxo- (SV5, MuV) (49-51) and rhabdoviruses (VSV, RV) (9, 52, 53). VBIs would require testing for antiviral activity towards these different viruses before clinical use, but nonetheless present a strong starting point for identifying therapeutics.

In this work we demonstrate antiviral activity of prazole compounds, with no detectable cell toxicity at effective concentrations, against two viruses that use different mechanisms for viral replication. Of particular note is that the viral genomes are very different, with HIV being RNA-based and HSV being DNA-based. That one compound works against viruses with such stark difference in viral life cycle types supports that these compounds have potential as a broad-spectrum antiviral agent for current and emerging viruses. This aspect gives this approach advantage over other potential broad-spectrum antivirals, such as remdesivir, which is targeted to RNA viruses, limiting its potential as a broad-spectrum antiviral (54).

Tsg101 binding to the proline-rich viral L-domains in Gag (3, 6, 7, 11, 14, 15) is required for virus particles to be released from cell membranes of infected cells. Tsg101 is a member of the ESCRT-I complex of proteins involved in cell endosomal sorting. The ESCRT-I complex recruits proteins from the ESCRT-III complex with AIP1 (19), which provides the mechanical means for scission of virus particles from cell membranes. Thus, blocking the PT/SAP L-domain sequence from interacting with 2host ESCRT complex causes the virus budding defect and three lines of independent evidence support this idea. First, drugs targeted to this specific interaction in HIV-1 cause virus budding defects in infected cells without detectable off-target effects (5). Second, a research group identified noncoding SNPs in the 5′ region of Tsg101 which correlate with viral load, implicating Tsg101-mediated viral particle release in disease progression (25). Third, viral infections activate a host innate immunity mechanism, through Interferon Stimulated Gene 15 (ISG15), that specifically disrupts virus budding complexes (1). In response to this immune system defense, many viruses encode enzymes that prevent or reverse ISG15 conjugation to cellular proteins to avoid the budding blockade (55-60). Taken together, this evidence indicates that targeting this interaction may lead to an effective antiviral strategy. Note that Pawliczek and Crump (31) have reported that HSV-1 production requires a functional ESCRT-III complex that could be independent of Tsg101 and Alix expression. However, there are multiple pathways to recruit ESCRT III proteins to functional virus budding complexes. For example, if we genetically replace the PT/SAP with the PPPPY L-domain in HIV-1 Gag, the virus still buds from cells independent of Tsg101 (10). Also, mutations of the HIV-1 L-domain in Gag causes a budding defect that can be rescued by overexpression of the specific ubiquitin ligase Nedd4L (12, 261 61). Nedd4L normally binds PPPPY motifs, which are absent from HIV-1 Gag. However, Nedd4 interacts with ESCRT-II proteins downstream from Tsg101, which in turn recruits the ESCRT-III proteins to the virus budding site (unpublished data). Thus, while Tsg101 is normally involved in recruiting the ESCRT-III complex, under stress its function can be replaced. This motivates our parallel investigation of small molecule inhibitors that target Nedd4's recruitment of the ESCRT-III complex. Independent of our work, Watanabe et al. (29) showed that release of a different herpes virus was susceptible to blockage by a prazole drug. They also used a HIV-1 Gag mutant bearing a disrupted PT/SAP motif (P7L-Gag) whose virus egress was independent of Tsg101 to demonstrate that release of this virus mutant was still blocked. This indicates that prazole drugs in particular are effective in blocking the budding process.

While the prazole analogs block the release of lenti and herpes viruses, the inhibition is manifested in different regions of the cell. The drugs block the release of HIV-1 at the outer cell membrane by preventing pinching of virus particles from the membrane (5). In contrast, herpesviruses, which assemble in the nucleus, appear to be first blocked at the passage of the virus through the nuclear membrane. Because the prazole drugs form a covalent bond to Tsg101, it strongly suggests that the ESCRT proteins are important for the herpesvirus particles to be released from the nucleus of the cell where they are formed. This is consistent with the recent report by Arii et al., (20) that the ESCRT-III protein complex mediates herpesvirus movement across the nuclear membrane and regulates its integrity. The finding that the prazole drugs cause a significant drop in total infectious herpesviruses reported here can be explained by the trapping of immature particles in the nucleus. This prevents them from migrating into the cytoplasm to exchange enveloped membranes, which makes them infectious. Also, the accumulation of the dense material in the nucleus observed in the electron micrographs suggests that prazole drugs may interfere with normal particle assembly in addition to blocking the passage of the particles through the nuclear membrane.

The use of prazoles represents an exciting potential case of repurposing existing drugs to act as antiviral therapeutic agents. Currently, omeprazole is marketed as a prodrug for treatment of acid reflux disease. Other prazole drugs are marketed for treatment of acid reflux disease in China, India, 290 and South Korea (Yi Li An, Adiza, Noltec, respectively) indicating reasonable bioavailability and a known clinical safety profile. The prodrugs are acid-activated into derivatives that form disulfide linkages with proton pumps (26, 62, 63). The prodrug, but not the charged sulfonamide derivative, can cross the plasma membrane barrier. The antiviral activity of tenatoprazole has been suggested to be the result of forming a covalent disulfide bond with Tsg101 (5). While the binding site for tenatoprazole is near the ubiquitin (Ub) binding pocket and not the L-domain binding site, biochemical and confocal imaging data independently demonstrated that tenatoprazole disrupts the binding of Tsg101 to the PT/SAP sequence (5). While the precise biochemical mechanism remains to be clarified, our FTS results support that it may be related to allosteric changes in Tsg101 after the drug forms its covalent linkage with Cys73. Previous reports did not detect off-target effects of the prazole drugs affecting Tsg101 metabolism inside of cells (5). A possible exception is noted in an epidemiological study in a peer reviewed preprint in the American Journal of Gastroenterology by B. Spiegel and colleagues (64). In these studies, there was a small correlation between SARS-CoV-2 infections and patients taking commercially available prazole drugs, such as omeprazole, for acid reflux disease. However, this does not preclude the use of prazole compounds described in this paper. The drugs used by the patients, such as omeprazole, have weak antiviral activity (Table 2). In contrast, Ilaprazole have potent antiviral activity. At a dose of 10 mg of ilaprazole/day, plasma concentrations are around 2 μg h/ml, which is within the range needed for antiviral activity (65). The prazoles we tested here also appear to be nontoxic to Vero, HeLa, and 293T cells at the concentrations used to inhibit budding of herpesviruses and HIV-1. To improve potency of the prazole drugs, we have synthesized 53 analogs of ilaprazole. Several of these appear to have stronger binding to Tsg101 detected by the FTS assay. We are now testing these analogs to see if they have a more potent antiviral activity than ilaprazole.

A recent report highlighted the potential of prazole compounds to have a therapeutic effect on SARS-CoV-2 when combined with remdesivir (66). However, the authors did not definitively identify the mechanism of action of the prazoles and also concluded that the potency of the prazole compound used, omeprazole, is too low to reach therapeutic levels in vivo. A mechanism posed by the authors is that the prazoles lead to an increase in lysosomal pH, which is the potential mechanism for lysosomotropic drugs such as chloroquine (67). In contrast to omeprazole, we hypothesize that ilaprazole may allow for therapeutic levels to be reached in vivo. In the case of ilaprazole, which is marketed in several Asian countries as discussed above, our strong in vitro results lay the foundation for a potential fast-track to broad-spectrum antiviral clinical testing, alone or in combination with other drugs, in these countries. We are currently working to determine if ilaprazole or our novel compounds have activity against SARS-CoV-2 with or in combination with remdesivir. This would further the potential broad-spectrum antiviral capacity of the prazole compounds described in this report.

Materials and Methods

Viruses, plasmids, cell lines. Herpes simplex virus-1 (Kos strain), Herpes simplex virus-2(A/B-G), HIV plasmid PR9-HIV-1Ba-L (Center For AIDS Research [CFAR] Lab). pET-28b vector (Novagen-EMD Millipore), ROSETTA 2 (DE3) pLysS E. coli competent cells (EMD Millipore), Vero cells and 293T cell lines.

Chemicals. Prazole Compounds: Rabeprazole, Lansoprazole, Omeprazole, Ilaprazole, Dexlansoprazole, Tenatoprazole, and Pantoprazole were from SelleckChem. 2-[(4-ethoxy-3-methylpridin-2-yl)methanesulfinyl]-1H-1,3benzodiazole, 2-[(3,5-dimethylpyridin-2-yl)methanesulfinyl]-5-methoxy-1H-1,3-benzodiazole, 4-methoxy-2-[[(5-methoxy-1H-1,3-benzodiazol-2-yl)sulfinyl] methyl]-3,5-dimethyl-1λ-pyridin-1-one were from MolPort. Esomeprazole was from Toronto Research Chemicals.

Purification of Tsg101 (1-145). N-terminally His6-tagged Tsg101 UEV domain (amino acids 1-145), called Tsg101-UEV, was encoded in a pET-28b vector (Novagen—EMD Millipore), which also included a thrombin protease cleavage site (His6-Thrombin Site—Tsg101, 1-145). Tsg101-UEV was grown in LB broth with Kanamycin (30 μg/ml) in ROSETTA 2 (DE3) pLysS E. coli competent cells (EMD Millipore) and induced with 1 mM IPTG at room temperature for 3 h. Bacteria were collected by centrifugation at 4,000 rpm for 10 min at 4 C. Bacteria were suspended in 50 ml binding buffer (20 mM Tris-HCl, pH 7.9, 0.5 M NaCl, 5 mM Imidazole) with 1 mM PMSF, 0.1% NP40, and a Protease Inhibitor Cocktail Tablet (Roche) and sonicated for 3.5 min on ice. The sonicate was spun at 9,000 rpm for 1 h at 4 C in a Sorvall centrifuge. The supernatant was collected and passed through a 1.5 ml Ni-NTA Agarose column. The column was washed with 20 mM Tris-HCl, pH 7.9, 0.5 M NaCl, 30 mM Imidazole wash buffer. The column was then equilibrated with TEV cleavage buffer followed by 50 units of thrombin in the same buffer (Novagene). The column flow was stopped and incubated at room temperature overnight. The cleaved protein was eluted with wash buffer, and the protein dialyzed in D-tube Dialyzer Maxi, MWCO 12-14 kDa (Novagene) overnight against 0.15 M NaCl, 0.1 M HEPES, pH 7.5 buffer. The protein was concentrated in a MicroSep Advanced Centrifugal Device, 12-14 kDa exclusion (Pall) for 1 h at 1,300 rpm). Protein concentration was determined with a Nano Drop Spectrophotometer at 280 nM. When the His tag was not removed, the protein was eluted from the Ni-NTA column with 20 mM Tris-HCl, pH 7.9, 0.5 M NaCl, 1 M Imidazole. The protein was evaluated by SDS-PAGE gel for purity

Fluorescence thermal shift (FTS) screening to identify small molecule binding to Tsg101-UEV. FTS using thermal shift elicited by the small molecule binding effect to protein stability. FTS monitors protein thermal denaturation using environment-sensitive dye Sypro® Orange which fluoresces when bound to hydrophobic surfaces, taking advantage of the changes in hydrophobic surface exposure in protein denaturation. Discovery of small molecule binding to target protein utilizes the observation that ligand binding affects protein thermal stability, and therefore can be detected through a shift in the protein's thermal denaturation (melting) temperature (Tₘ). We have employed FTS to reveal changes in thermodynamic properties of Tsg101 elicited by its interaction with a small molecule. The recombinant Tsg101 fragment (amino acids 1-145), prepared as described above in Materials and Methods (but without label) has a thermal unfolding profile suitable for using FTS as a primary screen assay in HTS. A fluorescence dye Sypro® Orange (Invitrogen) was used for assay detection. The dye is excited at 473 nm and has a fluorescence emission at 610 nm. The dye binds to hydrophobic regions of a protein that are normally buried in a native protein structure. When a protein is unfolded, the dye interacts with exposed hydrophobic surfaces and the fluorescence intensity increases significantly over that observed in aqueous solution. The Tsg101 fragment was premixed at a concentration of 2 μM with a 5× concentration of Sypro® Orange in HEPES buffer (100 mM HEPES, 150 mM NaCl, pH 7.5). Then 10 μl of the protein-dye mix was added to an assay plate and 10 to 50 nanoliters of compound, equal to 10 to 50 μM, were added with an acoustic transfer robot Echo550 (Labcyte, CA). The plate was shaken to ensure proper mixing, then sealed with an optical seal and centrifuged. The thermal scan was performed from 20 to 90° C. with a temperature ramp rate of 0.5° C./min. Fluorescence was detected on a real-time PCR machine CFX384 (Bio-Rad Laboratories). Comparison of the thermal denaturation profile for Tsg101-UEV in the presence and absence of tenatoprazole and other prazoles revealed destabilization of the native protein structure, indicating that the compound interacted with Tsg101-UEV.

Herpesvirus infection of Vero cells. Vero cells (0.8×10⁶ cells/well of a 6-well plate) were infected with HSV-1 or HSV-2 at a MOI of 0.1 pfu/cell in DMEM with 1% serum for two hours in the CO₂ incubator at 37 C. In one experiment looking at the effect of tenatoprazole on HSV-2 release from cells 24 and 48 h a MOI of 3 pfu/cell was used. The cell supernatants were aspirated and replaced with 1 ml (24 h) or 2 ml (48 h and 72 h) of DMEM with 1% serum with DMSO or different concentrations of drug (tenatoprazole, ilaprazole, or analogs) dissolved in DMSO. After 24 or 48 h incubation, the cell supernatant was collected and frozen at −80 C. Virus titer in the cell media fraction was determined by standard plaque assays using 10-fold serial dilutions of cell supernatants of Vero 393 cells and incubated for 48 h after which cells were fixed and stained to count the plaques (22). For determination of total virus (extracellular+cytoplasmic), virus infected cells were incubated for 24 h with and without drug presence, then the plate of cells were subjected to 3 cycles of freeze/thawing (−80 C/37 C) 30 min each prior to collecting the supernatant after centrifugation for measurement of total virus titer. Virus titer was measured by standard plaque assay as above. For analyzing the effect of benserazide (K21) at different concentrations on release of HSV-1 from VERO cells, experiments were repeated 4 times each and did not appear to affect release of virus from cells. In separate experiments, uninfected Vero cells were carried for 3 weeks in culture in the presence or absence of drugs (replaced every third day) and found to exhibit the same growth rate detected with a light microscope.

HIV-1 transfection of 293 T cells. 293T cells (American Type Culture Collection) were grown in a 24-well Clear Flat Bottom TC-treated Multiwell Cell Culture Plate using Dulbecco's modified Eagle's medium (Cellgro) containing fetal bovine serum (10%), 100 U/ml penicillin, 100 μg/ml streptomycin, and 292 μg/ml 1-glutamine (Cellgro). Cells were grown to 60-70% confluency at 37° C. and 5% CO₂ 408 prior to addition of drug treatment. Culture media was aspirated and replaced with media containing drug compound 7 hours prior to transfection of the plasmid encoding the HIV-1 genome. Transfection was done using reagent Polyethyleneimine (PEI, Polysciences). For production of virus particles, cells were transfected with pR9-HIV-1Ba-L plasmid. After 24 h and 48 h, tissue culture media was collected and passed through a 0.45-micron filter. Virus released from cells was quantified by media-associated p24 determined using fluorescently tagged CA targeting antibody (PerkinElmer) and equivalent amounts of p24 as standards.

Drug potency and cell toxicity. EC₅₀ calculations were determined by using AAT Bioquest's EC₅₀ calculator. Cell toxicity at different concentrations of drugs as indicated was determine using the Cell Proliferation Reagent WST-1 (Roche Diagnostics) or cellular 96® Aqueous One Reagent viability reagent according to manufacturer's instructions. For 293 T cells, the concentration of DMSO was 0.2% or less and assays were carried out with DMEM with 10% serum. Cell toxicity experiments were repeated twice.

Transmission electron Microscopy. Vero cells on glass cover slips were infected with HSV-2 at a MOI of 0.1 for two hours. Then 105 μM of tenatoprazole or 18 μM Ilaprazole was added and cells incubated for 24 hours. Tissue samples were fixed in 0.1 M sodium cacodylate buffer pH 7.3 containing 2% paraformaldehyde and 2.5% glutaraldehyde and post-fixed with 2% osmium tetroxide 427 in unbuffered aqueous solution. The samples were rinsed with distilled water, en bloc stained with 3% uranyl acetate, rinsed with distilled water, dehydrated in ascending grades of ethanol, transitioned with propylene oxide, embedded in the resin mixture of Embed 812 kit and cured in a 60° C. oven. Samples were sectioned on a Leica Ultracut UC6 ultramicrotome. 1 μm thick sections were collected and stained with Toluidine Blue O and 70 nm sections were collected on 200 mesh copper grids; thin sections were stained with uranyl acetate and Reynolds lead citrate. Transmission electron microscopy (TEM) was performed on a FEI Tecnai Spirit G2.

### REFERENCES

1. Seo E J, Leis J. 2012. Budding of Enveloped Viruses: Interferon-Induced ISG15 Antivirus Mechanisms Targeting the Release Process. Advances in virology 2012.
2. Carlton J G, Martin-Serrano J. 2007. Parallels between cytokinesis and retroviral budding: a role for the ESCRT machinery. Science 316:1908-1912.
3. Gottlinger H G, Dorfman T, Sodroski J G, Haseltine W A. 1991. Effect of mutations affecting the p6 gag protein on human immunodeficiency virus particle release. Proc Natl Acad Sci USA 460 88:3195-9.
4. Pincetic A, Medina G, Carter C, Leis J. 2008. Avian sarcoma virus and human immunodeficiency virus, type 1 use different subsets of ESCRT proteins to facilitate the budding process. Journal of Biological Chemistry 283:29822-29830.
5. Strickland M, Ehrlich L S, Watanabe S, Khan M, Strub M P, Luan C H, Powell M D, Leis J, Tjandra N, Carter C A. 2017. Tsg101 chaperone function revealed by HIV-1 assembly inhibitors. Nat Commun 8:1391.
6. Wills J W, Cameron C E, Wilson C B, Xiang Y, Bennett R P, Leis J. 1994. An assembly domain of the Rous sarcoma virus Gag protein required late in budding. J Virol 68:6605-18.
7. Xiang Y, Cameron C E, Wills J W, Leis J. 1996. Fine mapping and characterization of the Rous sarcoma virus Pr76gag late assembly domain. J Virol 70:5695-700.
8. Medina G, Pincetic A, Ehrlich L S, Zhang Y, Tang Y, Leis J, Carter C A. 2008. Tsg101 can replace Nedd4 function in ASV Gag release but not membrane targeting. Virology 377:30-38.
9. Taylor G M, Hanson P I, Kielian M. 2007. Ubiquitin depletion and dominant—negative VPS4 inhibit rhabdovirus budding without affecting alphavirus budding. Journal of virology 81:13631-475 13639.
10. Medina G, Zhang Y, Tang Y, Gottwein E, Vana M L, Bouamr F, Leis J,
Carter C A. 2005. The functionally exchangeable L domains in RSV and HIV-1 Gag direct particle release through pathways linked by Tsg101. Traffic 6:880-894.
11. VerPlank L, Agresta B, Grassa T, Kikonyogo A, Leis J, Carter C. 2001. Tsg101, the prototype of a class of dominant-negative ubiquitin regulators, binds human immunodeficiency virus type 1 Pr55Gag: the L domain is a determining of binding. Proc Natl Acad Sci USA 98:7724-7729.
12. Chung H Y, Morita E, von Schwedler U, Muller B, Krausslich H G, Sundquist W I. 2008. NEDD4L overexpression rescues the release and infectivity of human immunodeficiency virus type 1 constructs lacking PTAP and YPXL late domains. J Virol 82:4884-97.
13. Fujii K, Munshi U M, Ablan S D, Demirov D G, Soheilian F, Nagashima K, Stephen A G, Fisher R J, Freed E O. 2009. Functional role of Alix in HIV-1 replication. Virology 391:284-292.
14. Garrus J E, von Schwedler U K, Pornillos O W, Morham S G, Zavitz K H, Wang H E, Wettstein D A, Stray K M, Cote M, Rich R L, Myszka D G, Sundquist W I. 2001. Tsg101 and the vacuolar protein sorting pathway are essential for HIV-1 budding. Cell 107:55-65.
15. Goff A, Ehrlich L S, Cohen S N, Carter C A. 2003. Tsg101 control of human immunodeficiency virus type 1 Gag trafficking and release. J Virol 77:9173-82.
16. Martin-Serrano J, Yaravoy A, Perez-Caballero D, Bieniasz P D. 2003. Divergent retroviral late-budding domains recruit vacuolar protein sorting factors by using alternative adaptor proteins. Proceedings of the National Academy of Sciences 100:12414-12419.
17. Pornillos O, Alam S L, Rich R L, Myszka D G, Davis D R, Sundquist W I. 2002. Structure and functional interactions of the Tsg101 UEV domain. The EMBO journal 21:2397-2406.
18. von Schwedler U K, Stuchell M, Muller B, Ward D M, Chung H Y, Morita E, Wang H E, Davis T, He G P, Cimbora D M, Scott A, Krausslich H G, Kaplan J, Morham S G, Sundquist W I. 2003. The protein network of HIV budding. Cell 114:701-13.
19. Strack B, Calistri A, Craig S, Popova E, Göttlinger HG. 2003. AIP1/ALIX is a binding partner for HIV-1 p6 and EIAV p9 functioning in virus budding. Cell 114:689-699.
20. Arii J, Watanabe M, Maeda F, Tokai-Nishizumi N, Chihara T, Miura M, Maruzuru Y, Koyanagi N, Kato A, Kawaguchi Y. 2018. ESCRT-III mediates budding across the inner nuclear membrane and regulates its integrity. Nat Commun 9:3379.
21. Lee C P, Liu P T, Kung H N, Su M T, Chua H H, Chang Y H, Chang C W, Tsai C H, Liu F T, Chen M R. 2012. The ESCRT machinery is recruited by the viral BFRF1 protein to the nucleus-associated membrane for the maturation of Epstein-Barr Virus. PLoS Pathog 8:e1002904.
22. Kuang Z, Seo E J, Leis J. 2011. Mechanism of inhibition of retrovirus release from cells by interferon-induced gene ISG15. Journal of virology 85:7153-7161.
23. Pincetic A, Leis J. 2009. The Mechanism of Budding of Retroviruses From Cell Membranes. Adv Virol 2009:6239691-6239699.
24. Pincetic A, Kuang Z, Seo E J, Leis J. 2010. The interferon-induced gene ISG15 blocks retrovirus release from cells late in the budding process. J Virol 84:4725-36.
25. Bashirova A A, Bleiber G, Qi Y, Hutcheson H, Yamashita T, Johnson R C, Cheng J, Alter G, Goedert J J, Buchbinder S, Hoots K, Vlahov D, May M, Maldarelli F, Jacobson L, O'Brien S J, Telenti A, Carrington M. 2006. Consistent effects of TSG101 genetic variability on multiple outcomes of exposure to human immunodeficiency virus type 1. J Virol 80:6757-63.
26. Shin J M, Kim N. 2013. Pharmacokinetics and pharmacodynamics of the proton pump inhibitors. J Neurogastroenterol Motil 19:25-35.
27. Luan C-H, Light S H, Dunne S F, Anderson W F. 2014. Ligand screening using fluorescence thermal shift analysis (FTS), p 263-289, Structural Genomics and Drug Discovery. Springer.
28. Pantoliano M W, Petrella E C, Kwasnoski J D, Lobanov V S, Myslik J, Graf E, Carver T, Asel E, Springer B A, Lane P, Salemme F R. 2001. High-density miniaturized thermal shift assays as a general strategy for drug discovery. J Biomol Screen 6:429-40.
29. Watanabe S M, Ehrlich L S, Strickland M, Li X, Soloveva V, Goff A J, Stauft C B, Bhaduri—McIntosh S, Tjandra N, Carter C. 2020. Selective Targeting of Virus Replication by Proton Pump Inhibitors. Sci Rep 10:4003.
30. Lee S K, Longnecker R. 1997. The Epstein-Barr virus glycoprotein 110 carboxy-terminal tail domain is essential for lytic virus replication. Journal of virology 71:4092-4097.
31. Pawliczek T, Crump C M. 2009. Herpes simplex virus type 1 production requires a functional ESCRT-III complex but is independent of TSG101 and ALIX expression. Journal of virology 83:11254-11264.
32. Calistri A, Sette P, Salata C, Cancellotti E, Forghieri C, Comin A, Gottlinger H, Campadelli—Fiume G, Palù G, Parolin C. 2007. Intracellular trafficking and maturation of herpes simplex virus type 1 gB and virus egress require functional biogenesis of multivesicular bodies. Journal of virology 81:11468-11478.
33. Calistri A, Munegato D, Toffoletto M, Celestino M, Franchin E, Comin A, Sartori E, Salata C, Parolin C, Palu G. 2015. Functional Interaction Between the ESCRT-I Component TSG101 and the HSV-1 Tegument Ubiquitin Specific Protease. Journal of cellular physiology 230:1794-540 1806.
34. Crump C M, Yates C, Minson T. 2007. Herpes simplex virus type 1 cytoplasmic envelopment requires functional Vps4. Journal of virology 81:7380-7387.
35. Tandon R, AuCoin D P, Mocarski E S. 2009. Human cytomegalovirus exploits ESCRT machinery in the process of virion maturation. J Virol 83:10797-807.
36. Perez M, Craven R C, de la Torre J C. 2003. The small RING finger protein Z drives arenavirus budding: implications for antiviral strategies. Proc Natl Acad Sci USA 100:12978-83.
37. Urata S, Noda T, Kawaoka Y, Yokosawa H, Yasuda J. 2006. Cellular factors required for Lassa virus budding. Journal of virology 80:4191-4195.
38. Ariumi Y, Kuroki M, Maki M, Ikeda M, Dansako H, Wakita T, Kato N. 2011. The ESCRT system is required for hepatitis C virus production. PloS one 6.
39. Corless L, Crump C M, Griffin S D, Harris M. 2010. Vps4 and the ESCRT—III complex are required for the release of infectious hepatitis C virus particles. Journal of General Virology 91:362-372.
40. Han Z, Lu J, Liu Y, Davis B, Lee M S, Olson M A, Ruthel G, Freedman B D,
Schnell M J, Wrobel J E. 2014. Small-molecule probes targeting the viral PPxY-host Nedd4 interface block egress of a broad range of RNA viruses. Journal of virology 88:7294-7306.
41. Harty R N, Brown M E, Wang G, Huibregtse J, Hayes F P. 2000. A PPxY motif within the VP40 protein of Ebola virus interacts physically and functionally with a ubiquitin ligase: implications for filovirus budding. Proceedings of the National Academy of Sciences 97:13871-13876.
42. Lu J, Han Z, Liu Y, Liu W, Lee M S, Olson M A, Ruthel G, Freedman B D, Harty R N. 2014. A host-oriented inhibitor of Junin Argentine hemorrhagic fever virus egress. Journal of virology 562 88:4736-4743.
43. Madara J J, Han Z, Ruthel G, Freedman B D, Harty R N. 2015. The multifunctional Ebola virus VP40 matrix protein is a promising therapeutic target. Future virology 10:537-546.
44. Martin-Serrano J, Zang T, Bieniasz P D. 2001. HIV-1 and Ebola virus encode small peptide motifs that recruit Tsg101 to sites of particle assembly to facilitate egress. Nature medicine 7:1313-1319.
45. Silvestri L S, Ruthel G, Kallstrom G, Warfield K L, Swenson D L, Nelle T,
Iversen P L, Bavari S, Aman M J. 2007. Involvement of vacuolar protein sorting pathway in Ebola virus release independent of TSG101 interaction. J Infect Dis 196 Suppl 2:S264-70.
46. Timmins J, Schoehn G, Ricard-Blum S, Scianimanico S, Vernet T, Ruigrok R W, Weissenhorn W. 2003. Ebola virus matrix protein VP40 interaction with human cellular factors Tsg101 and Nedd4. J Mol Biol 326:493-502.
47. Urata S, Noda T, Kawaoka Y, Morikawa S, Yokosawa H, Yasuda J. 2007. Interaction of Tsg101 with Marburg virus VP40 depends on the PPPY motif, but not the PT/SAP motif as in the case of Ebola virus, and Tsg101 plays a critical role in the budding of Marburg virus-like particles induced by VP40, N P, and G P. J Virol 81:4895-9.
48. Lambert C, Doring T, Prange R. 2007. Hepatitis B virus maturation is sensitive to functional inhibition of ESCRT-III, Vps4, and 72-adaptin. Journal of virology 81:9050-9060.
49. Li M, Schmitt P T, Li Z, McCrory T S, He B, Schmitt A P. 2009. Mumps virus matrix, fusion, and nucleocapsid proteins cooperate for efficient production of virus-like particles. Journal of virology 83:7261-7272.
50. Schmitt A P, Leser G P, Morita E, Sundquist W I, Lamb R A. 2005. Evidence for a new viral late—domain core sequence, FPIV, necessary for budding of a paramyxovirus. J Virol 79:2988-97.
51. Schmitt A P, Leser G P, Waning D L, Lamb R A. 2002. Requirements for budding of paramyxovirus simian virus 5 virus-like particles. J Virol 76:3952-64.
52. Irie T, Harty R N. 2005. L-domain flanking sequences are important for host interactions and efficient budding of vesicular stomatitis virus recombinants. Journal of virology 79:12617-589 12622.
53. Wirblich C, Tan G S, Papaneri A, Godlewski P J, Orenstein J M, Harty R N, Schnell M J. 2008. PPEY motif within the rabies virus (R V) matrix protein is essential for efficient virion release and R V pathogenicity. Journal of virology 82:9730-9738.
54. Gordon C J, Tchesnokov E P, Feng J Y, Porter D P, Gotte M. 2020. The antiviral compound remdesivir potently inhibits RNA-dependent RNA polymerase from Middle East respiratory syndrome coronavirus. J Biol Chem 295:4773-4779.
55. Arguello M D, Hiscott J. 2007. Ub surprised: viral ovarian tumor domain proteases remove ubiquitin and ISG15 conjugates. Cell Host Microbe 2:367-9.
56. Frias-Staheli N, Giannakopoulos N V, Kikkert M, Taylor S L, Bridgen A, Paragas J, Richt J A, Rowland R R, Schmaljohn C S, Lenschow D J. 2007. Ovarian tumor domain-containing viral proteases evade ubiquitin- and ISG15-dependent innate immune responses. Cell host & microbe 2:404-416.
57. Harty R N, Pitha P M, Okumura A. 2009. Antiviral activity of innate immune protein ISG15. Journal of innate immunity 1:397-404.
58. Vana M L, Tang Y, Chen A, Medina G, Carter C, Leis J. 2004. Role of Nedd4 and ubiquitination of Rous sarcoma virus Gag in budding of virus-like particles from cells. Journal of virology 78:13943-13953.
59. Yuan W, Aramini J M, Montelione G T, Krug R M. 2002. Structural basis for ubiquitin-like ISG 15 protein binding to the NS1 protein of influenza B virus: a protein-protein interaction function that is not shared by the corresponding N-terminal domain of the NS1 protein of influenza A virus. Virology 304:291-301.
60. Yuan W, Krug R M. 2001. Influenza B virus NS1 protein inhibits conjugation of the interferon (IFN)-induced ubiquitin-like ISG15 protein. EMBO J 20:362-71.
61. Usami Y, Popov S, Popova E, Gottlinger HG. 2008. Efficient and specific rescue of human immunodeficiency virus type 1 budding defects by a Nedd4-like ubiquitin ligase. J Virol 82:4898-907.
62. Kakinoki B, Ono C, Yamazaki N, Chikamatsu N, Wakatsuki D, Uchiyama K, Morinaka Y. 1999. General pharmacological properties of the new proton pump inhibitor (+/−)-5-methoxy-2-[[(4-methoxy-3,5-dimethylpyrid-2-yl)methyl]sulfi nyl]-1H-imidazo [4,5-b] pyridine. Methods Find Exp Clin Pharmacol 21:179-187.
63. Shin J M, Sachs G. 2002. Restoration of acid secretion following treatment with proton pump inhibitors. Gastroenterology 123:1588-97.
64. Chistopher V. Almario W D C, Brennan M. R. Spiegel. 2020. Increased Risk of COVID-19 Among Users of Proton Pump Inhibitors. American Journal of Gastroenterology Preprint.
65. de Bortoli N, Martinucci I, Giacchino M, Blandizzi C, Marchi S, Savarino V, Savarino E. 2013. The pharmacokinetics of ilaprazole for gastro-esophageal reflux treatment. Expert Opin Drug Metab Toxicol 9:1361-9.
66. Bojkova D, McGreig J E, McLaughlin K-M, Masterson S G, Widera M, Kraehling V, Ciesek S, Wass M N, Michaelis M, Cinatl J N. 2020. SARS-CoV-2 and SARS-CoV differ in their cell tropism and drug sensitivity profiles. bioRxiv.
67. Al-Bari MAA. 2017. Targeting endosomal acidification by chloroquine analogs as a promising strategy for the treatment of emerging viral diseases. Pharmacol Res Perspect 5:e00293.

### Example 2

Abstract

We developed a novel strategy targeting budding of lenti- (HIV), herpes, and other enveloped viruses to control their infections by blocking the process of virus release from cells. This approach is based on scientific findings from our lab that established budding as a natural target to control enveloped virus release and the identification of a class of small molecule inhibitors that disrupt this process in the nmolar range. We refer to these inhibitors as viral budding inhibitors (VBIs). This discovery was first reported in our Nature Communications paper, along with the mechanism of how these inhibitors block the budding process of HIV-1. (See Strickland et al., “Tsg101 chaperone function revealed by HIV-1 assembly inhibitors,” Nat Commun. 2017 Nov. 9; 8(1):1291.doi: 10.1038/s41467-017-01426-2; US Publication No. 20200368258; US Publication No. 20190209589; US Publication No. 20170095485; and US Publication No. 20140179637; the contents of which are incorporated herein by reference in their entireties).

There are no therapies to date that target this crucial budding process, so these are first-in-class drugs, capable of blocking the release of multiple viruses from cells. They would be much more resistant to the selection of drug resistant viruses. In addition, they may promote more efficient removal of virus-infected cells because particles accumulate on the cell surface. This would represent a very significant advance in treatment of AIDS patients. The same drugs quantitatively block the release of HSV-1/2 from cells indicating that these drugs are broad-based antiviral agents.

Applications

Applications of the disclosed subject matter may include, but are not limited to preventing enveloped viruses from budding from cells, slowing down the spread of viruses from infected cells, and giving the host immune system time to clear the infection.

Advantages

The disclosed subject matter relates to small molecule inhibitors that block the release of infectious enveloped viruses that use a common mechanism to bud from cells. There are no comparable small molecule inhibitors currently available

Description

Small molecule inhibitors that prevent viruses from recruiting ESCRT III proteins required to provide the mechanical means of scission of the cell envelope membrane to release viruses from cells. We have now synthesized 53 novel prazole analogs designed by Jim Audia based on commercially available prazole drugs and our activity data against Tsg101. Of these, 8 ViraL Budding Inhibitors (VBIs) are confirmed hits that have tighter binding to Tsg101 than the most potent commercial drug we have tested. Moreover, we have now confirmed that 4 of these hit compounds inhibit the budding of HIV-1 from 293T cells in the nmolar range.

### Example 3

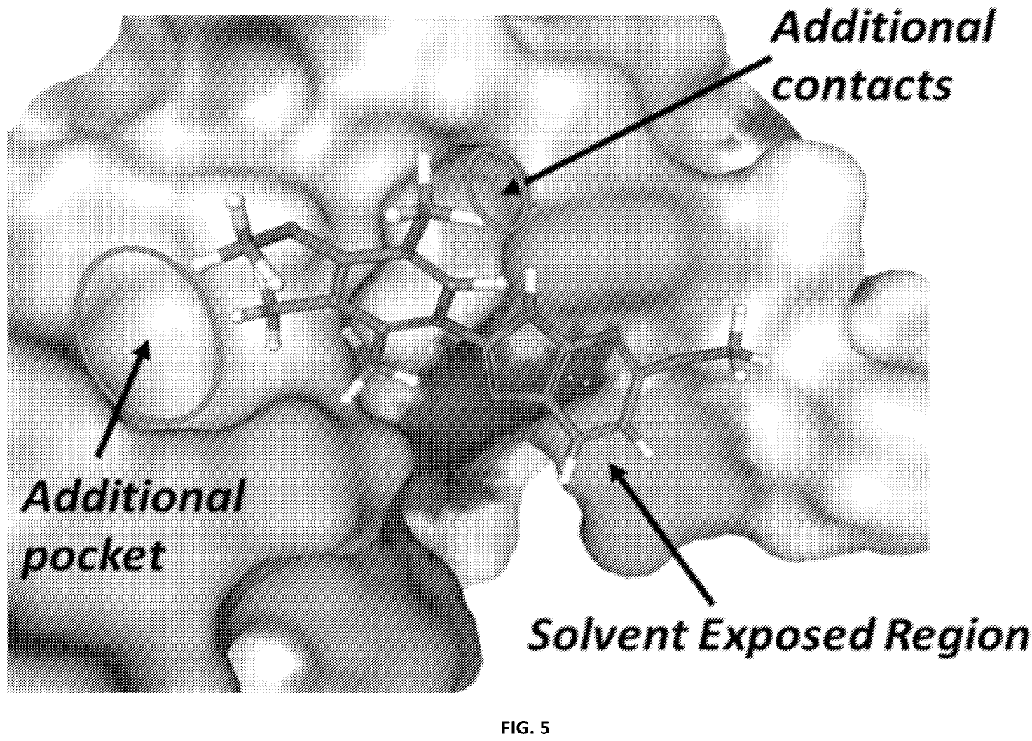

To improve our chances of identifying a more potent inhibitor, we are synthesizing a library of analogs of prazoles working with medicinal chemists in the ChemCore lab on our Northwestern University Evanston campus. This established facility has both the capacity and experience to produce the required analogs for further testing. Ilaprazole has reasonable physiochemical characteristics, has several clear structural areas for modification, and again we have access to a NMR structure with covalently linked N16 in the 2017 Nature Communications paper (47) and others (37) to use as a guide (FIG. 5) The NMR structure of N16 bound to Tsg101 shows a large unoccupied pocket adjacent to the methyl group of the pyridine ring. This is the site where Ilaprazole has the additional ring structure that improved potency over tenatoprazole. The library to be synthesized will include analogs with diverse substituents at this position (see Scheme 1) to try to capture additional favorable contacts with the protein and improve binding and potency. Substitutions at R⁴ and R⁵ are also proposed because they are predicted to affect other contacts between the prazole drug and the surface of Tsg101 based upon our analysis of commercially available analogs (see circles in FIG. 5).

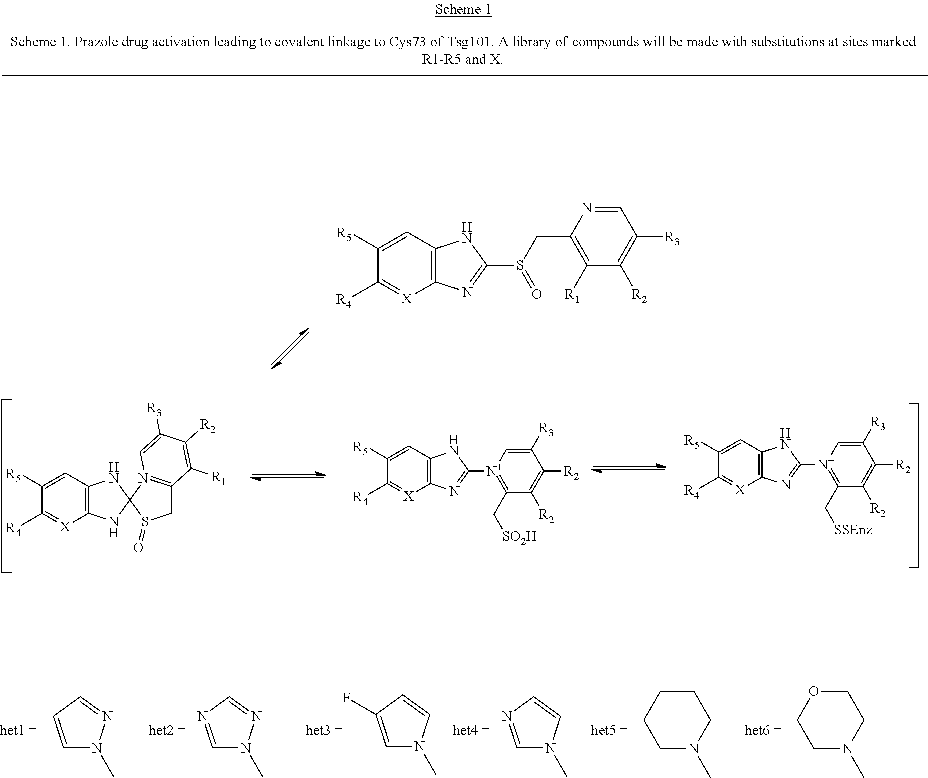

Structure-Activity Relationship (SAR) studies to date suggest that binding (evidenced by thermal shift) and phenotypic potency (direct effects on viral budding) are influenced by a combination of steric and electronic effects as might be expected based upon the currently accepted mechanism of enzyme inactivation by ‘prazoles’. To advance our detailed understanding of this SAR, we specifically propose the synthesis and biological evaluation of a small matrix/library (84 analogs) of compounds which systematically explore substituent effects at six positions of the parent prazole (R¹⁻⁵═H, X═CH) scaffold. Compounds can be rapidly evaluated in the thermal shift assay in the High Throughput Analysis Lab. They can also be counter screened as proton pump inhibitors to further determine both the primary SAR drivers and opportunities to introduce selectivity over the pH elevating effects. (Since we are optimizing against the Tsg101 target, we may find substitutions that selectively decrease activity towards cell proton pumps (71).)

In Scheme 1 above, the top structure substituents are R¹ either H or CH₃; R² either H, OCH₃, OCH₂CH₃, or O(CH₂)₃OCH₃; R³ either H or CH₃; R⁴ either H, OCH₃, N-pyrrolo, or heterocycles: het1-het6 (as shown in the bottom of Scheme 1), and R⁵ either H, OCH₃ or N-pyrrolo. The advantages of such a design are several-fold, including allowing for rapid and efficient parallel synthesis, which will reduce costs, application of QSAR (quantitative SAR) methodology to better understand the interplay of steric and electronic effects, and the ability to rapidly explore both primary activity and selectivity in parallel. The results from the initial library synthesis and evaluation will also allow for the prioritization of positions/vectors for further optimization, including refinement of ADME properties, although the series already benefits from the prior progression of analogs into human clinical use. Based upon the screening of the library, we will combine the best substitutions that enhance potency into a single compound that we will evaluate biologically for potency, toxicity, and off target effects, including effects on proton pumps.

The Fluorescence Thermal Sensitivity assay was used to measure the binding of different concentrations of compounds (0.5 to 20 mM) to purified Tsg101 (2-145). Tₘ is a measure of the thermal stability of the protein. Eight synthesized compounds showed a lower Tₘ than RC #2.

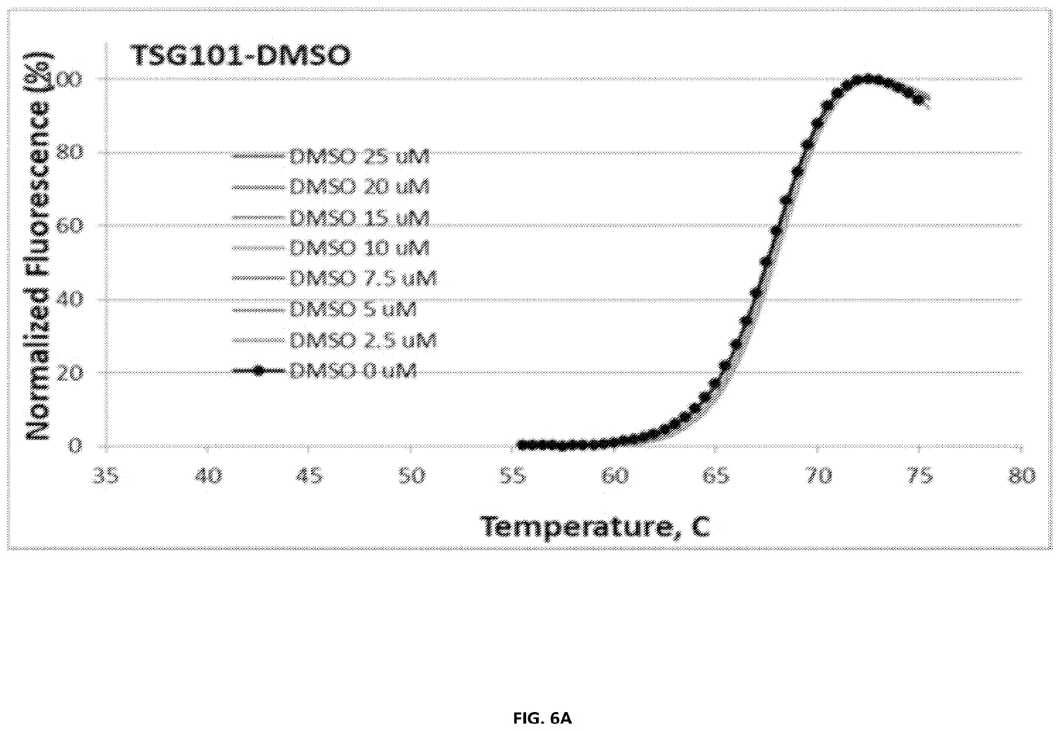

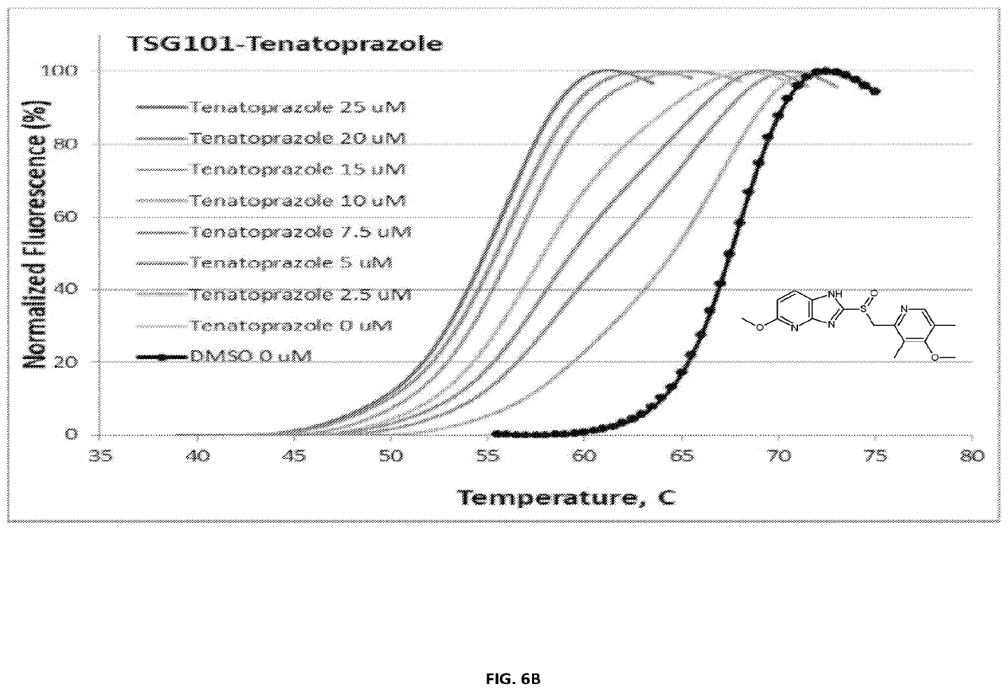

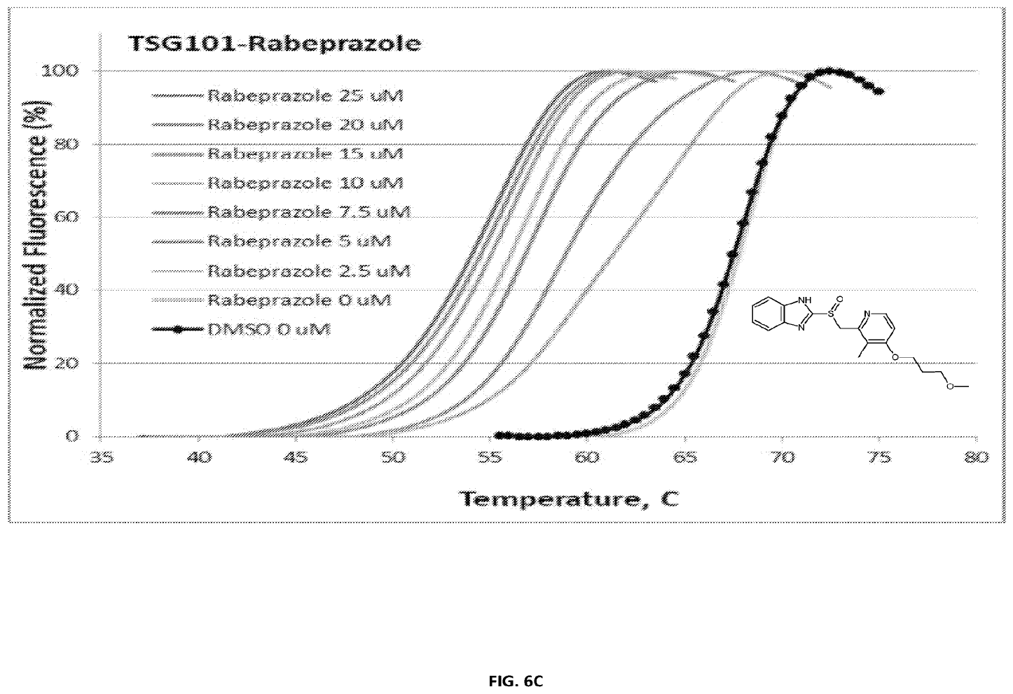

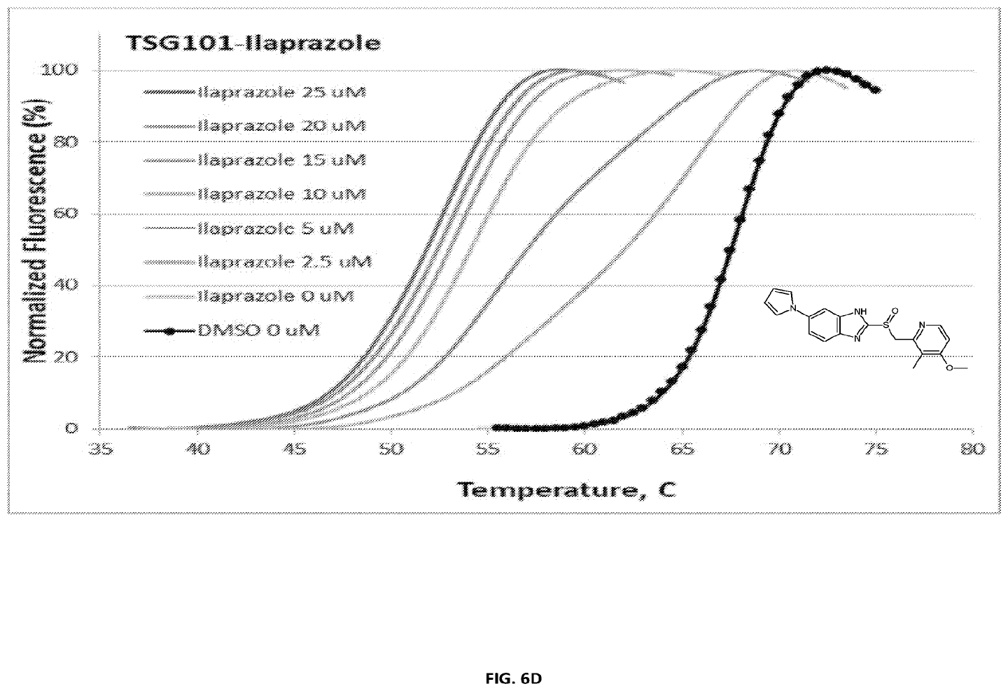

FIG. 6A-6C are graphs showing the fluorescence thermal sensitivity of reference compounds Ilaprazole, Rabeprazole, and Tenatoprazole. Table 5 shows the Tₘ of two reference compounds.

TABLE 5
Tₘ of two reference compounds.

| Reference compounds | tenatoprazole | Ilaprazole |
|---|---|---|
| Tₘ (EC₅₀) | 57° C. | 55° C. |

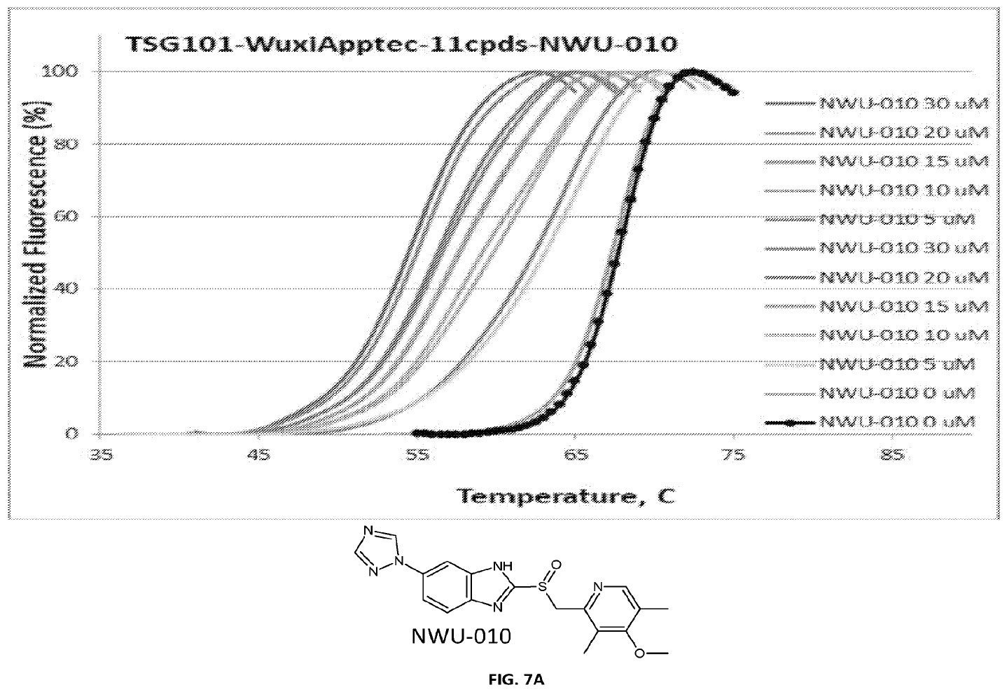

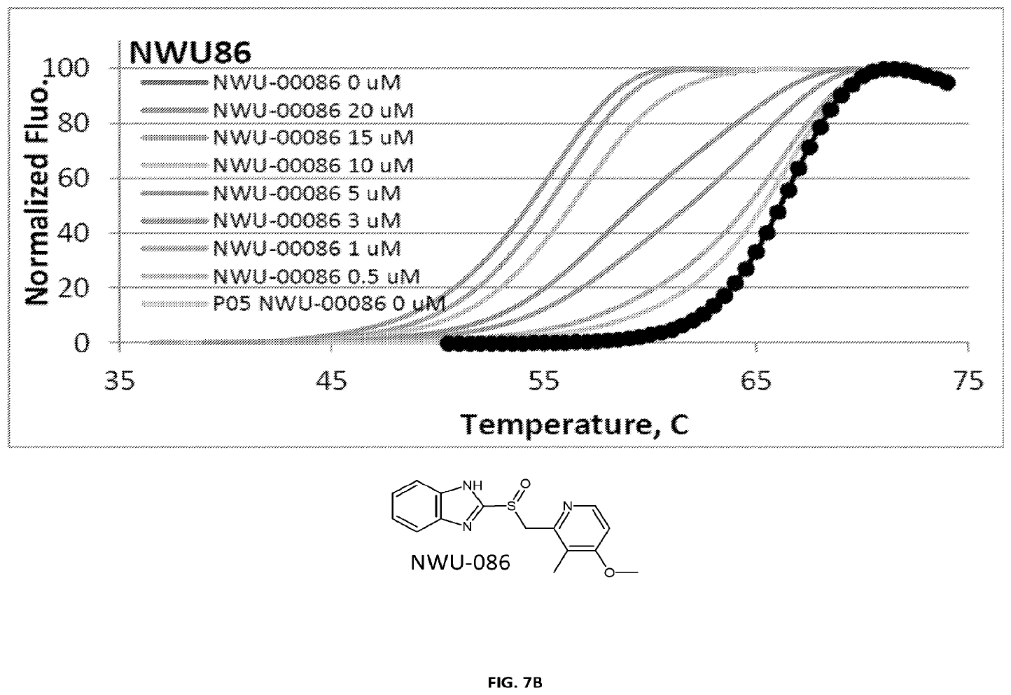

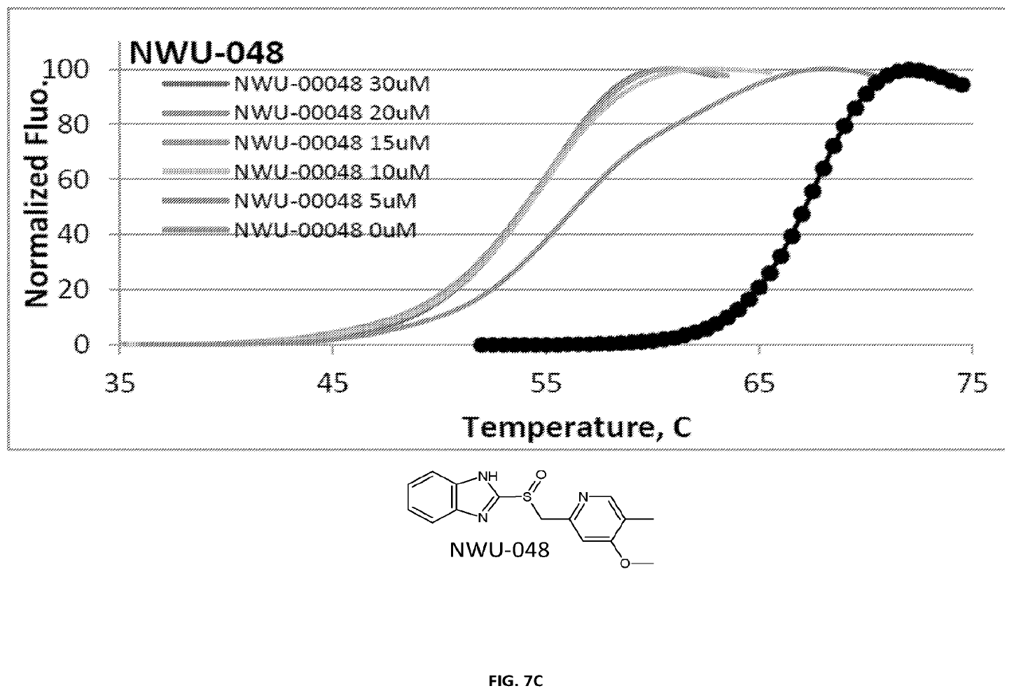

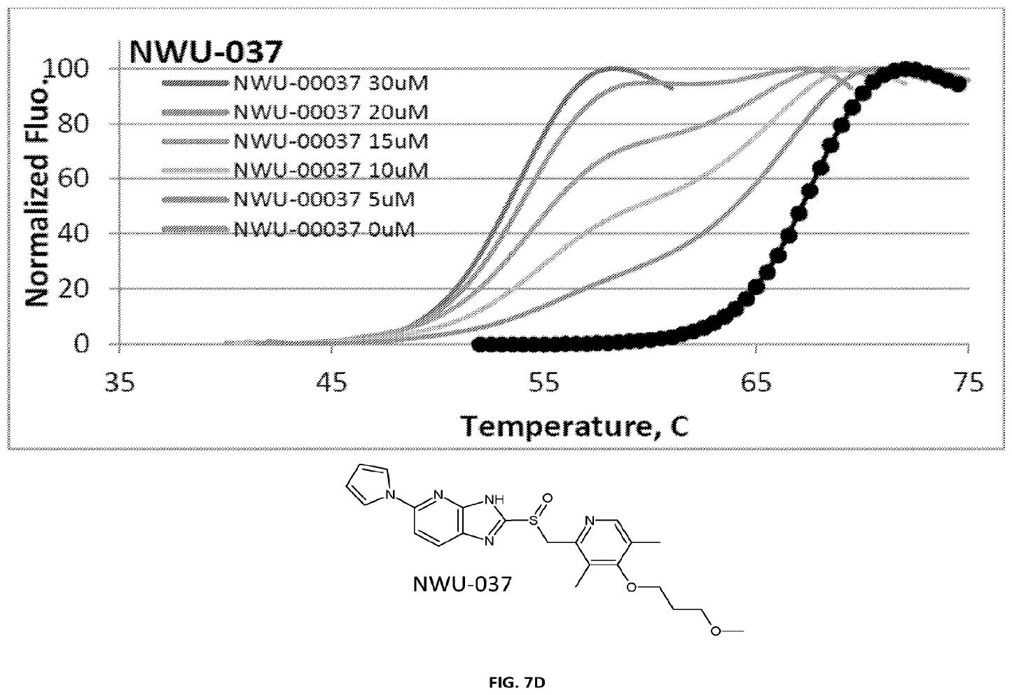

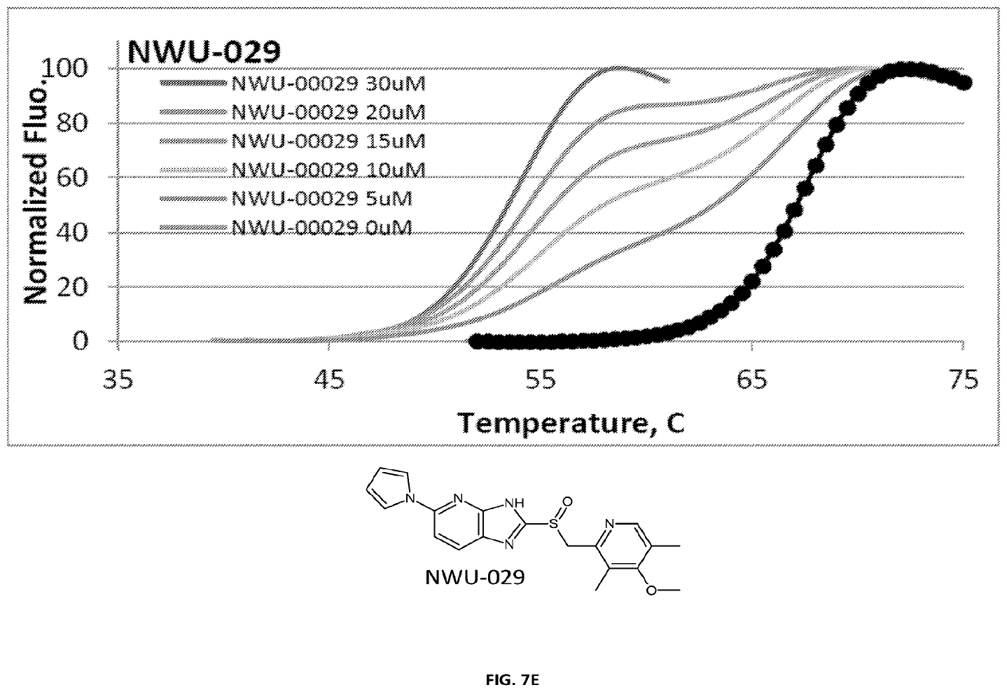

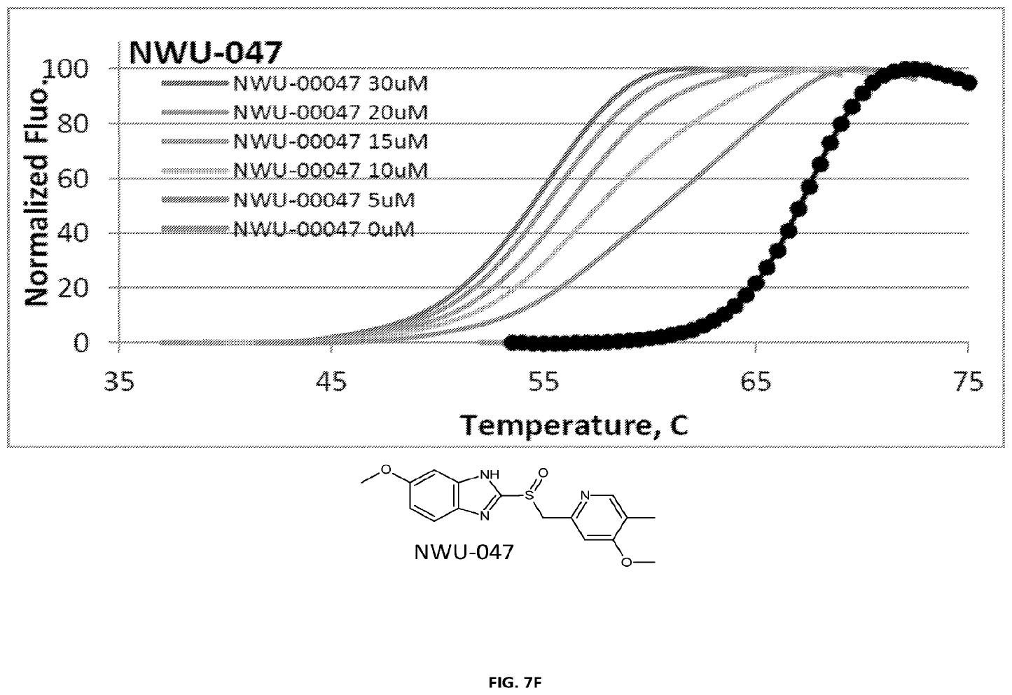

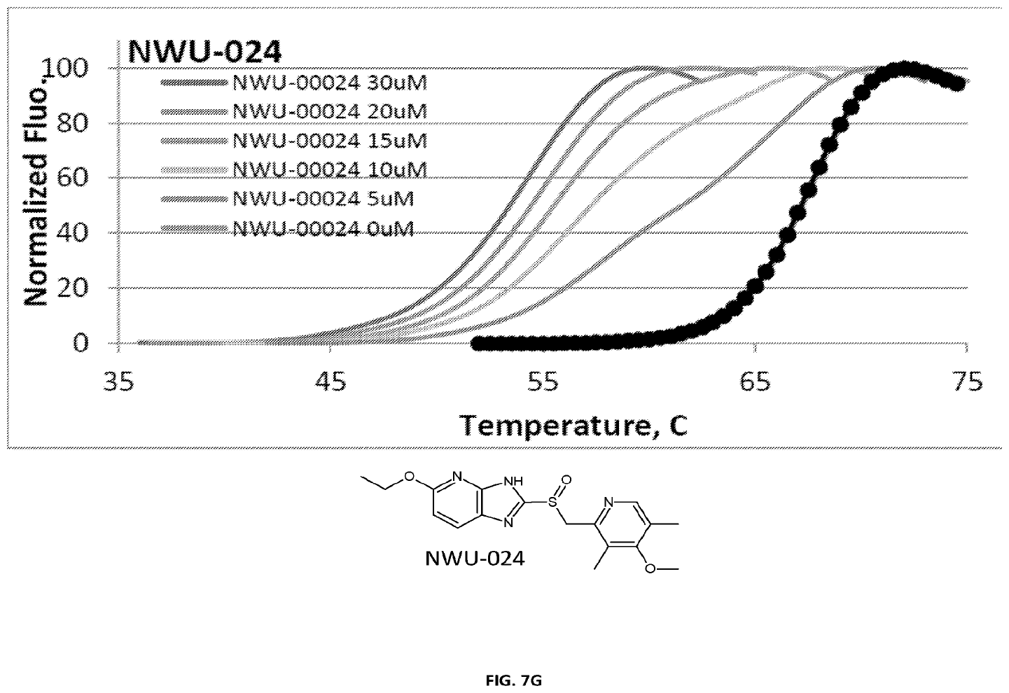

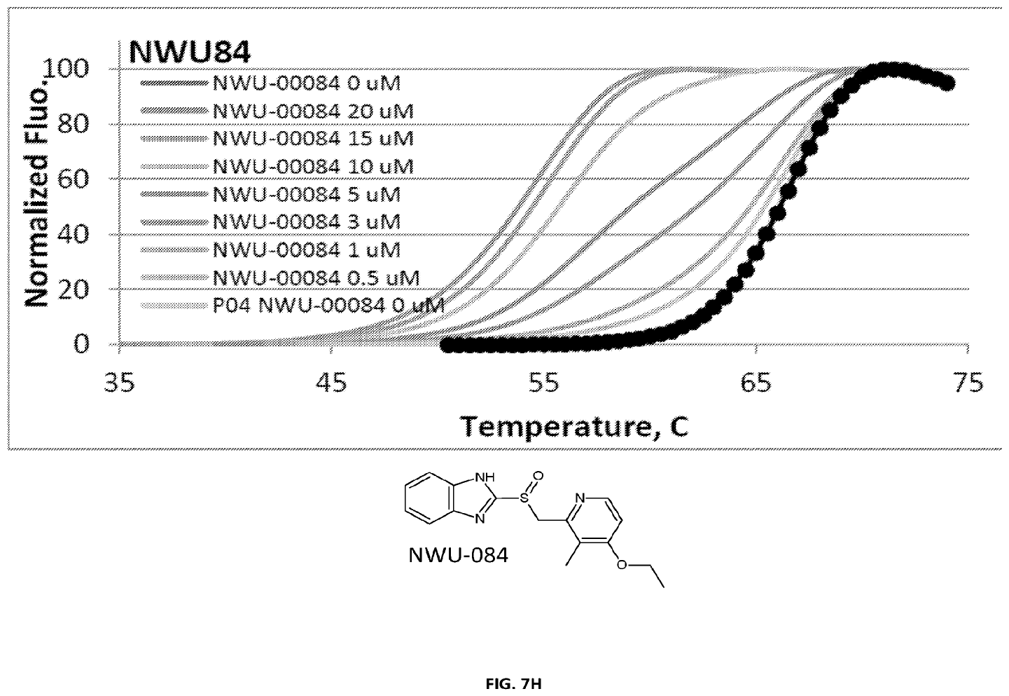

FIG. 7A-H are graphs showing the fluorescence thermal sensitivity of selected synthetic compounds. Table 6 shows the Tₘ of selected compounds.

TABLE 6
Tₘ of selected compounds.

| | Synthesized Compounds | | | | | | | |
|---|---|---|---|---|---|---|---|---|
| | 10 | 24 | 29 | 37 | 47 | 48 | 84 | 86 |
| Tₘ | 54° | 54° | 53.5° | 53.5° | 54° | 54° | 54.5° | 54.5° |
| (EC₅₀) | C. | C. | C. | C. | C. | C. | C. | C. |

### Example 4

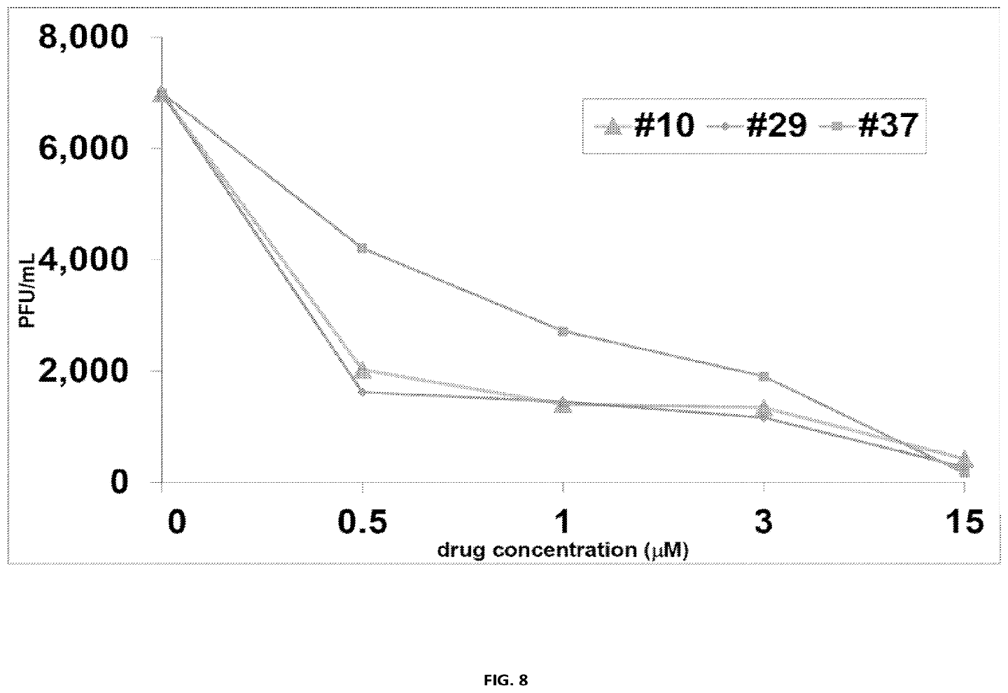

FIG. 8 shows that compounds 10, 29, and 37 inhibited the release of HIV-1 in the nanomolar range.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

We claim:

1. A compound of the following formula or a pharmaceutically acceptable salt thereof: 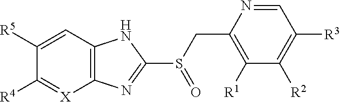
wherein,
X is CH or N,
R¹ is H or CH₃;
R² is H, methoxy, ethoxy, or methoxypropoxy;
R³ is H or CH₃;
R⁴ is H, methoxy, N-pyrrolo, pyrazole, triazole, 3-fluoro-pyrrole, imidazole, piperidine, or morpholine; and
R⁵ is H, methoxy, or N-pyrrolo.

2. The compound of claim 1, wherein X is CH.

3. The compound of claim 1, wherein X is N.

4. The compound of claim 1, wherein R¹ is CH₃.

5. The compound of claim 1, wherein R² is methoxy.

6. The compound of claim 1, wherein R³ is CH₃.

7. A compound selected from the group consisting of:

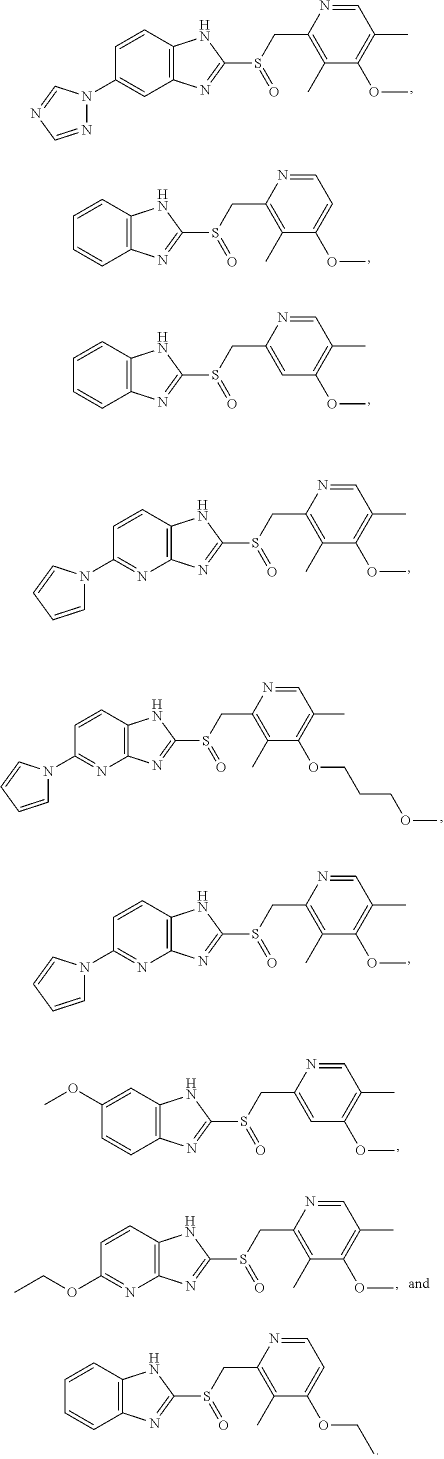

8. A pharmaceutical composition comprising an effective amount of the compound of claim 1 for treating a viral infection by an enveloped virus in a patient and a suitable pharmaceutical excipient.

9. A method of treating an infection by an enveloped virus in a patient, the method comprising administering to the patient a pharmaceutical composition comprising the compound of claim 1.

10. The method of claim 9, wherein the enveloped virus is selected from the group consisting of Lassa fever virus, lymphocytic choriomeningitis virus, Ebola virus, Marburg virus, hepatitis B virus, herpes simplex virus type 1, herpes simplex virus type 2, cytomegalovirus, simian virus type 5, mumps virus, avian sarcoma leucosis virus, human immunodeficiency virus type 1, human T-lymphotrophic virus type 1, equine infectious anemia virus, vesicular stomatitis virus, rabies virus, coronavirus (e.g., a human coronavirus such as sudden acute respiratory syndrome coronavirus (SARS-CoV) or sudden acute respiratory syndrome coronavirus 2 (SARS-CoV-2), and combinations thereof.

11. The method of claim 9, wherein the compound has antiviral activity against the enveloped virus selected from (i) inhibiting formation of an associative complex, (ii) disrupting formation of an associative complex, and (iii) both of (i) and (ii), wherein the associative complex comprises an L-domain motif of the enveloped virus and at least one cellular polypeptide, or fragment thereof, capable of binding the L-domain motif of the enveloped virus.

12. The method of claim 11, wherein the L-domain motif comprises at least one of a PY-motif or a PTAP-motif.

13. The method of claim 11, wherein the L-domain motif comprises at least one amino acid sequence selected from the group consisting of SEQ ID NOS: 1-22.

14. The method of claim 11, wherein the at least one cellular polypeptide comprises an ESCRT complex protein.

15. The method of claim 14, wherein the ESCRT component protein comprises at least one member selected from a Nedd 4-related family peptide or a fragment thereof, TSG101 or a fragment thereof, and combinations thereof.

16. The method of claim 14, wherein the ESCRT component protein comprises at least one amino acid sequence selected from the group consisting of SEQ ID NOS: 24, 29, 32 and 33.

17. A method of inhibiting release of an enveloped virus from a cell, the method comprising contacting the cell with a compound of claim 1.

18. The method of claim 17, wherein the enveloped virus is selected from the group consisting of Lassa fever virus, lymphocytic choriomeningitis virus, Ebola virus, Marburg virus, hepatitis B virus, herpes simplex virus type 1, herpes simplex virus type 2, cytomegalovirus, simian virus type 5, mumps virus, avian sarcoma leucosis virus, human immunodeficiency virus type 1, human T-lymphotrophic virus type 1, equine infectious anemia virus, vesicular stomatitis virus, rabies virus, coronavirus, and combinations thereof.

19. The method of claim 17, wherein the enveloped virus is a human coronavirus.

20. The method of claim 1, wherein the enveloped virus is sudden acute respiratory syndrome coronavirus (SARS-CoV) or sudden acute respiratory syndrome coronavirus 2 (SARS-CoV-2).

21. The method of claim 1, wherein the compound has antiviral activity against the enveloped virus selected from (i) inhibiting formation of an associative complex, (ii) disrupting formation of an associative complex, and (iii) both of (i) and (ii), wherein the associative complex comprises an L-domain motif of the enveloped virus and at least one cellular polypeptide, or fragment thereof, capable of binding the L-domain motif of the enveloped virus.

22.-26. (canceled)
